SEQUENCE LISTING (28 sequences)

SEQ ID NO: 1 - protein, 184 aa, Homo sapiens
  Human BCMA (Isoform1)
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - protein, 135 aa, Homo sapiens
  Human BCMA (Isoform2)
MLQMAGQCSQNEYFDSLLHACIPCQLRCSSNTPPLTCQRYCNARSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 3 - protein, 183 aa, Macaca fascicularis
  Cynomolgus BCMA
MLQMARQCSQNEYFDSLLHDCKPCQLRCSSTPPLTCQRYCNASMTNSVKGMNAILWTCLGLSLIISLAVFVLTFLLRKMSSEPLKDEFKNTGSGLLGMANIDLEKGRTGDEIVLPRGLEYTVEECTCEDCIKNKPKVDSDHCFPLPAMEEGATILVTTKTNDYCNSLSAALSVTEIEKSISAR

SEQ ID NO: 4 - protein, 185 aa, Mus musculus
  Murine BCMA
MAQQCFHSEYFDSLLHACKPCHLRCSNPPATCQPYCDPSVTSSVKGTYTVLWIFLGLTLVLSLALFTISFLLRKMNPEALKDEPQSPGQLDGSAQLDKADTELTRIRAGDDRIFPRSLEYTVEECTCEDCVKSKPKGDSDHFFPLPAMEEGATILVTTKTGDYGKSSVPTALQSVMGMEKPTHTR

SEQ ID NO: 5 - protein, 7 aa, Artificial Sequence
  2265-F02, CDR-H1, Chothia
GFNISAP

SEQ ID NO: 6 - protein, 5 aa, Artificial Sequence
  2265-F02, CDR-H1, Kabat
APGIH

SEQ ID NO: 7 - protein, 6 aa, Artificial Sequence
  2265-F02, CDR-H2, Chothia
NPAGGY

SEQ ID NO: 8 - protein, 17 aa, Artificial Sequence
  2265-F02, CDR-H2, Kabat
FINPAGGYTDYADSVKG

SEQ ID NO: 9 - protein, 13 aa, Artificial Sequence
  2265-F02, CDR-H3
DYIRQYWTYVLDY

SEQ ID NO: 10 - protein, 11 aa, Artificial Sequence
  trastuzumab, CDR-L1
RASQDVNTAVA

SEQ ID NO: 11 - protein, 7 aa, Artificial Sequence
  trastuzumab, CDR-L2
SASFLYS

SEQ ID NO: 12 - protein, 9 aa, Artificial Sequence
  trastuzumab, CDR-L3
QQHYTTPPT

SEQ ID NO: 13 - protein, 122 aa, Artificial Sequence
  2265-F02, VH
EVQLVESGGGLVQPGGSLRLSCAASGFNISAPGIHWVRQAPGKGLEWVGFINPAGGYTDYADSVKGRFTISADTSKNTAYLQMNSLRAEDTAVYYCARDYIRQYWTYVLDYWGQGTLVTVSS

SEQ ID NO: 14 - protein, 107 aa, Artificial Sequence [sequence omitted]

SEQ ID NO: 15 - protein, 452 aa, Artificial Sequence
  2265-F02, Heavy Chain
EVQLVESGGGLVQPGGSLRLSCAASGFNISAPGIHWVRQAPGKGLEWVGFINPAGGYTDYADSVKGRFTISADTSKNTAYLQMNSLRAEDTAVYYCARDYIRQYWTYVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1356 bp, Artificial Sequence
  2265-F02, Heavy Chain
gaagttcagttagtggaatcaggcggcggtttagttcaaccaggcggttcattgcgtctgtcatgcgcggcttccggtttcaacatcagtgcgcctgggatccattgggtgcgtcaggccccaggcaagggtctggagtgggtcggttttatcaatcctgctggcggttataccgactatgcggactctgtgaagggtcgcttcaccattagcgcggatacctcgaagaatacggcgtatttacagatgaattccctgcgtgcagaggacactgccgtctactattgtgcgcgcgattacattcggcagtactggacctacgttcttgactactggggccagggtacgctggtcaccgtgtcgtcggcgtcaaccaagggtccgtcggtttttccgctggcgccgtcgtcaaaatctacgtccggtggtaccgccgctctgggttgcctggttaaagactactttccggagccggtcacggtttcgtggaactctggtgccctgacttctggcgtccacacgttcccagccgttttgcagtcatccggtctgtagtcgttgtcctctgtggtcacggtgccgtcatcgtctctgggcacccaaacctatatctgcaatgtcaaccacaaaccgtccaatacgaaagttgacaaaaaagtcgagccgaaatcttgcgacaagacccacacgtgccctccgtgcccggcaccggaactgctgggcggtccgtcggtgttcctgttcccgccgaagccgaaagatactctgatgatctcacgtaccccggaagtcacgtgtgttgttgttgacgtgtcacacgaagatccagaggtgaaattcaattggtatgtggacggtgtcgaagtgcataatgccaaaaccaaaccgcgcgaggaacagtacaactccacctaccgcgtcgtgtcggtgttgaccgtcctgcatcaagactggctgaacggtaaagagtacaagtgcaaggtttcaaataaggcactgcctgcgccgattgaaaagaccatctctaaggcaaagggccagccgcgtgagccacaggtgtataccctgccgccgtcgcgtgaagaaatgaccaagaaccaagtttcactgacgtgtctggtcaagggcttttatccgtccgatattgcggtggagtgggagtctaatggccagccggaaaacaattacaaaacgactccgccggtgctggattccgacggttcgtagttcctgtattccaagctgaccgttgacaaatcacgttggcagcaaggcaacgttttttcttgttcggtaatgcacgaagcgctgcacaatcattacacccagaaatcactgtcgttgtctccgggcaaa

SEQ ID NO: 17 - protein, 214 aa, Artificial Sequence
  2265-F02, Light Chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 642 bp, Artificial Sequence
  2265-F02, Light Chain
gacattcaaatgacccagtctccgtcgtcactgtccgcatccgttggcgaccgcgttaccatcacgtgccgtgcgtcgcaagatgtgaacaccgccgtggcgtggtatcagcaaaaaccgggcaaagctccgaagctgctgatctattcagcctctttcctgtactcgggtgttccgtcccgtttctcaggctctcgctcgggtacggatttcaccctgactatttcttcactgcaaccggaagattttgcgacgtactactgtcagcagcattacacgactccgccgacctttggtcagggtaccaaggtcgagattaagcgtaccgtggctgcaccatccgtgtttatcttccctccgtctgatgagcagctgaaatccggtacggcgtcggtcgtctgcttgctgaataacttctatccgcgtgaagcgaaggtgcaatggaaggttgacaatgccctgcagtcaggtaactcccaagagtctgttaccgaacaagattcgaaagactcaacctactccctgtcttcgacgctgacgttgtccaaagcggactatgagaaacacaaggtttacgcatgtgaagtgacccaccagggcctgtcatctccggtcaccaaatcatttaatcgcggtgagtgc

SEQ ID NO: 19 - protein, 330 aa, Homo sapiens
  Human IgG1 HC Constant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 107 aa, Homo sapiens [sequence omitted]

SEQ ID NO: 21 - protein, 323 aa, Mus musculus
  Mouse IgG1 HC Constant
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 22 - protein, 107 aa, Mus musculus
  Mouse IgG LC Constant Ckappa
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - protein, 108 aa, Artificial Sequence
  Kappa LC
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 106 aa, Artificial Sequence
  Lambda LD
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 20 aa, Artificial Sequence
  FlagHis Tag
GSGDYKDDDDKGSGHHHHHH

SEQ ID NO: 26 - protein, 15 aa, Artificial Sequence
  Linker
GGGGSGGGGSGGGGS

SEQ ID NO: 27 - protein, 11 aa, Artificial Sequence
  Linker
AAGSDQEPKSS

SEQ ID NO: 28 - protein, 30 aa, Artificial Sequence
  Linker
  MISC_FEATURE (16)..(30): “Gly Gly Gly Gly Ser” may or may not be present
GGGGSGGGGSGGGGSGGGGSGGGGSGGGGS

(19) United States
(12) Patent Application Publication
(10) Pub. No.: US 2022/0323599 A1
(43) Pub. Date: Oct. 13, 2022

(54) ANTI-BCMA ANTIBODY CONJUGATE, COMPOSITIONS COMPRISING THE SAME, AND METHODS OF MAKING AND USING THE SAME

(71) Applicant: CELGENE CORPORATION, Summit, NJ (US)

(72) Inventors: John LEE, San Francisco, CA (US); Ryan STAFFORD, Emeryville, CA (US); Alice YAM, Tiburon, CA (US); Xiaofan LI, Fremont, CA (US); Abigail YU, San Jose, CA (US); Amandeep GAKHAL, San Mateo, CA (US)

(73) Assignee: CELGENE CORPORATION, Summit, NJ (US)

(21) Appl. No.: 17/608,097

(22) PCT Filed: May 1, 2020
(86) PCT No.: PCT/US2020/031067

§ 371 Date: Nov. 1, 2021

Related U.S. Application Data
(60) Provisional application No. 62/843,226, filed on May 3, 2019.

(51) Int. Cl. A61K 47/68; C07K 16/28; A61P 35/02

(52) U.S. Cl. CPC A61K 47/6849; A61K 47/6803; C07K 16/2878; C07K 16/2896; A61K 47/6867; A61K 47/6889; A61P 35/02

(57) ABSTRACT

The present disclosure relates to antibody conjugates with binding specificity for BCMA (BCMA) and its isoforms and homologs, and compositions comprising the antibody conjugates, including pharmaceutical compositions. Also provided are methods of producing the antibody conjugates and compositions as well as methods of using the antibody conjugates and compositions, such as in therapeutic and diagnostic methods.

### CROSS-REFERENCE TO RELATED APPLICATIONS

This application claims the benefit of U.S. Provisional Application No. 62/843,226, filed May 3, 2019, which is incorporated by reference herein in its entirety.

### REFERENCE TO SEQUENCE LISTING SUBMITTED ELECTRONICALLY

This application incorporates by reference a Sequence Listing submitted with this application as text file entitled 14247-525-228_SEQ_LISTING.txt created on Apr. 28, 2020 and having a size of 30,207 bytes.

### FIELD OF THE INVENTION

Provided herein are antibody conjugates with binding specificity for B-cell maturation antigen (BCMA) and compositions comprising the antibody conjugates, including pharmaceutical compositions, methods of producing the conjugates, and methods of using the conjugates and compositions for therapy. The conjugates and compositions are useful in methods of treatment and prevention of cell proliferation and cancer, methods of detection of cell proliferation and cancer, and methods of diagnosis of cell proliferation and cancer. The conjugates and compositions are also useful in methods of treatment, prevention, detection, and diagnosis of autoimmune diseases and infectious diseases.

### BACKGROUND

B-cell maturation antigen (BCMA) is a member of the tumor necrosis factor (TNF) receptor superfamily which recognizes B-cell activating factor. The protein in humans is encoded by the tumor necrosis factor receptor superfamily member 17 (TNFRSF17) gene and is preferentially expressed in mature B lymphocytes.

BCMA plays an important role in regulating B-cell maturation and differentiation into plasma cells. It is closely related to BAFF receptor (BAFF-R) and transmembrane activator and calcium modulator and cyclophilin ligand interactor (TACI). While BCMA, BAFF-R, and TACI are type III transmembrane proteins that promote B-cell survival at distinct stages of development, BCMA is expressed exclusively in B-cell lineage cells, such as, for example, plasmablasts and differentiated plasma cells (Avery et al. (2003) 1 Clin. Invest. 112(2):286-297; O'Connor et al. (2004) J Exp. Med. 199(1):91-98). It is selectively induced during plasma cell differentiation, which occurs concurrently with loss of BAFF-R expression in the differentiated cells (Darce et al. (2007) J. Immunol. 178(9):5612-5622). BCMA expression appears to support the survival of normal plasma cells and plasmablasts but is typically absent on naïve and most memory B cells. Thus, it does not appear to be needed for overall B-cell homeostasis but is required for optimal survival of long-lived plasma cells in the bone marrow (O'Connor et al. (2004) supra; Xu, S. and K. P. Lam (2001)Mol. Cell. Biol. 21(12):4067-4074).

In multiple myeloma, BCMA has been shown to be universally and widely expressed in malignant plasma cells at elevated levels; however, it is typically undetected on normal human tissues except for plasma cells. Due to its selective expression as a cell-surface receptor on multiple myeloma cell lines, BCMA can potentially be targeted in therapies to treat multiple myeloma. BCMA expression is also associated with leukemia and lymphoma. Accordingly, there is a need for improved methods of targeting and/or modulating the activity of BCMA. Given the specific expression of BCMA on plasma cells and lower expression in non-cancer tissue, there is a need for improved therapeutics that can specifically target cells and tissues that express or overexpress BCMA. Antibody conjugates to BCMA could be used to deliver therapeutic or diagnostic payload moieties to target cells expressing BCMA for the treatment or diagnosis of such diseases.

### SUMMARY

Provided herein are antibody conjugates that selectively bind B-cell maturation antigen (BCMA). The antibody conjugates comprise an antibody, that binds BCMA, linked to one or more payload moieties. The antibody is linked to the payload by way of a linker. BCMA antibodies are described in detail herein, as are useful payload moieties, and useful linkers.

In another aspect, provided are compositions comprising the antibody conjugates. In some embodiments, the compositions are pharmaceutical compositions. Any suitable pharmaceutical composition may be used. In some embodiments, the pharmaceutical composition is a composition for parenteral administration. In a further aspect, provided herein are kits comprising the antibody conjugates or pharmaceutical compositions.

In another aspect, provided herein are methods of using the anti-BCMA antibody conjugates. In some embodiments, the methods are methods of delivering one or more payload moieties to a target cell or tissue expressing BCMA. In some embodiments, the methods are methods of treatment. In some embodiments, the methods are diagnostic methods. In some embodiments, the methods are analytical methods. In some embodiments, the antibody conjugates are used to treat a disease or condition. In some aspects, the disease or condition is selected from a cancer, autoimmune disease, and infection.

In some embodiments, the antibody conjugates bind human BCMA. In some embodiments, the antibody conjugates also bind homologs of human BCMA. In some aspects, the antibody conjugates also bind cynomolgus monkey and/or mouse BCMA.

In certain embodiments, provided herein is an antibody conjugate according to the formula:

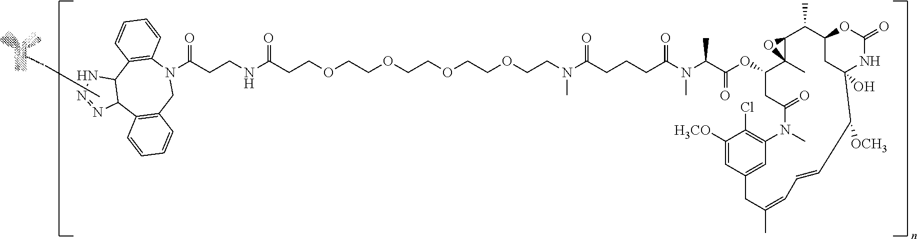

wherein n is from 1 to 4; the antibody comprises a V_{H} region of SEQ ID NO: 13, and a V_{L} region of SEQ ID NO: 14; the antibody further comprises a heavy chain constant region comprising residue of p-azidomethyl-phenylalanine substituting at each of sites HC-F404 and HC-Y180 according to the EU numbering scheme; and each structure within the brackets of the formula is bonded to the antibody at one of the p-azidomethyl-phenylalanine residues. In other embodiments, the antibody comprises (i) a V_{H} region comprising a CDR1 comprising SEQ ID NO SEQ ID NO: 5 or 6; a CDR2 comprising SEQ ID NO: 7 or 8; a CDR3 comprising SEQ ID NO: 9; and (ii) a V_{L} comprising a CDR1 comprising SEQ ID NO: 10; a CDR2 comprising SEQ ID NO: 11; and a CDR3 comprising SEQ ID NO: 12. In more specific embodiments, of the antibody conjugate, n is 1, 2, 3 or 4. In particular embodiments, the antibody conjugate further comprises at least one constant region domain. For example, in specific embodiments, the antibody conjugate comprise a human constant region domain, e.g. In yet other specific embodiments, the antibody conjugate comprises a constant region domain that comprises a human IgG1 heavy chain contant region, a human IgG1 kappa light chain region, or a human IgG1 heavy chain constant region and a human IgG1 kappa light chain region. In a more specific embodiment of the antibody conjugate, the constant region comprises a sequence selected from SEQ ID NO: 19 and 20, or both SEQ ID NO: 19 and SEQ ID NO: 20. In other embodiments, the antibody conjugate comprises a heavy chain that comprises the amino acid sequence of SEQ ID NO: 15. For example, the antibody conjugate may comprise a heavy chain that comprises the amino acid sequence of SEQ ID NO: 15, wherein each of the amino acids corresponding to HC-F404 and HC-Y180 according to the EU numbering scheme have been substituted for a p-azidomethyl-phenylalanine residue. In other embodiments, the antibody conjugate comprises a light chain that comprises the amino acid sequence of SEQ ID NO: 17. In yet other embodiments, the antibody conjugate comprises a heavy chain that comprises the amino acid sequence of SEQ ID NO:15 and a light chain that comprises the amino acid sequence of SEQ ID NO: 17. For example, the antibody conjugate may comprise a heavy chain that comprises the amino acid sequence of SEQ ID NO:15 and a light chain that comprises the amino acid sequence of SEQ ID NO: 17, wherein each of the amino acids corresponding to heavy chain (HC)-F404 and HC-Y180 according to the EU numbering scheme have been substituted for a p-azidomethyl-phenylalanine residue.

In certain embodiments of any of the antibody conjugates provided herein, the antibody is a monoclonal antibody. In certain embodiments of any of the antibody conjugates provided herein, the antibody is an IgA, an IgD, an IgE, an IgG, or an IgM. In certain embodiments of any of the antibody conjugates provided herein, the antibody is humanized or human. In certain embodiments of any of the antibody conjugates provided herein, the antibody is aglycosylated. In certain embodiments of any of the antibody conjugates provided herein, the antibody is an antibody fragment, e.g, an Fv fragment, a Fab fragment, a F(ab′)2 fragment, a Fab′ fragment, an scFv (sFv) fragment, or an scFv-Fc fragment. In certain embodiments of any of the antibody conjugates provided herein the antibody specifically binds human BCMA and cynomolgus BCMA. In certain embodiments of any of the antibody conjugates provided herein, the antibody specifically binds human BCMA and mouse BCMA.

Further provided herein are kits comprising any of the antibody conjugates provided herein, and instructions for use of the antibody conjugate. In a specific embodiment, the antibody conjugate is lyophilized. In another specific embodiment, the kit further comprises a fluid for reconstitution of the lyophilized antibody.

Further provided herein are pharmaceutical compositions comprising any of the antibody conjugates provided herein, and a pharmaceutically acceptable carrier.

Further provided herein are methods of treating or preventing a disease or condition in a subject in need thereof, comprising administering to the subject an effective amount of any of the antibody conjugates provided herein, or a pharmaceutical composition of any of the antibody conjugates provided herein. In certain embodiments, the disease or condition is a cancer. In certain embodiments, the disease or condition is leukemia or lymphoma. In certain embodiments, the disease or condition is multiple myeloma. In specific embodiments, said multiple myeloma is Stage I, Stage II, or Stage III according to the International Staging System or the Revised International Staging System. In certain embodiments, said multiple myeloma is newly-diagnosed multiple myeloma. In other embodiments, said multiple myeloma is relapsed or refractory multiple myeloma.

Further provided herein are methods of diagnosing a disease or condition in a subject in need thereof, comprising administering to the subject an effective amount of any of the antibody conjugates provided herein. In certain embodiments, the disease or condition is a cancer. In certain embodiments, the disease or condition is leukemia or lymphoma. In certain embodiments, the disease or condition is multiple myeloma. In specific embodiments, said multiple myeloma is Stage I, Stage II, or Stage III according to the International Staging System or the Revised International Staging System. In certain embodiments, said multiple myeloma is newly-diagnosed multiple myeloma. In other embodiments, said multiple myeloma is relapsed or refractory multiple myeloma.

### BRIEF DESCRIPTION OF THE FIGURES

FIG. 1 provides a comparison of the Kabat and Chothia numbering systems for CDR-H1. Adapted from Martin A. C. R. (2010). Protein Sequence and Structure Analysis of Antibody Variable Domains. In R. Kontermann & S. Dübel (Eds.), Antibody Engineering vol. 2 (pp. 33-51). Springer-Verlag, Berlin Heidelberg.

FIG. 2 is a graph illustrating body weight changes in mice implanted with ARP-1 multiple myeloma tumors after being administered a single dose of different BCMA antibody-drug conjugates as disclosed herein.

FIGS. 3A and 3B are graphs illustrating tumor growth curves and tumor size in mice implanted with ARP-1 multiple myeloma tumors after being administered a single dose of different BCMA antibody-drug conjugates as disclosed herein.

FIG. 4 is a graph illustrating body weight changes in mice implanted with MM.1S multiple myeloma cells after being administered a single dose of different BCMA antibody-drug conjugates as disclosed herein.

FIG. 5 is a graph illustrating Kaplan-Meier survival plots in mice implanted with MM.1S multiple myeloma cells after being administered a single dose of different BCMA antibody-drug conjugates as disclosed herein.

FIG. 6 is a graph illustrating Kaplan-Meier survival plots in mice implanted with MM.1S multiple myeloma cells after being administered a single dose of a BCMA antibody-drug conjugate, Daratumumab, Velcade, or different combinations thereof as disclosed herein.

FIGS. 7A-7C are graphs illustrating survival plots in mice implanted with MM.1S multiple myeloma cells after being administered a single dose of a BCMA antibody-drug conjugate along with either Daratumumab or Velcade as disclosed herein

FIGS. 8A and 8B are graphs illustrating a Kaplan-Meier survival plot and a survival plot of mice implanted with MM.1S multiple myeloma cells after being administered a single dose of a BCMA antibody-drug conjugate at different concentrations as disclosed herein.

FIG. 9 is a graph illustrating body weight changes in mice implanted with ARP-1 multiple myeloma tumors after being administered a single dose of a BCMA antibody-drug conjugate at different doses as disclosed herein.

FIGS. 10A and 10B are graphs illustrating tumor growth curves and tumor size in mice implanted with ARP-1 multiple myeloma tumors after being administered a single dose of a BCMA antibody-drug conjugate at different doses as disclosed herein.

FIG. 11 is a graph illustrating the average DAR of Conjugate 4 over time in PBS, human, mouse, and cynomolgus plasma.

FIG. 12 provides graphs illustrating cell binding of Conjugate 4 and Conjugate 1 to cells expressing human BCMA, BAFF-R, and TACI receptors.

### DETAILED DESCRIPTION OF THE EMBODIMENTS

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Green & Sambrook, Molecular Cloning: A Laboratory Manual 4^{th} ed. (2012), Cold Spring Harbor Laboratory Press, Cold Spring Harbor, N.Y.; and Ausubel et al., Current Protocols in Molecular Biology, John Wiley & Sons. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value ±10%, ±5%, or ±1%. In certain embodiments, the term “about” indicates the designated value ±one standard deviation of that value.

The term “combinations thereof” includes every possible combination of elements to which the term refers to. For example, a sentence stating that “if α₂ is A, then α₃ is not D; as is not S; or α₆ is not S; or combinations thereof” includes the following combinations when α₂ is A: (1) α₃ is not D; (2) as is not S; (3) α₆ is not S; (4) α₃ is not D; as is not S; and α₆ is not S; (5) α₃ is not D and as is not S; (6) α₃ is not D and α₆ is not S; and (7) as is not S and α₆ is not S.

The terms “BCMA” and “B-cell maturation antigen” are used interchangeably herein. BCMA is also known by synonyms, including BCM, tumor necrosis factor receptor superfamily member 17 (“TNFRSF17”), CD269, TNFRSF13A, and TNF receptor superfamily member 17, among others. Unless specified otherwise, the terms include any variants, isoforms and species homologs of human BCMA that are naturally expressed by cells, or that are expressed by cells transfected with a BCMA or BCMA gene. BCMA proteins include, for example, human BCMA isoform 1 (SEQ ID NO: 1) and human BCMA isoform 2 (SEQ ID NO: 2). In some embodiments, BCMA proteins include cynomolgus monkey BCMA (SEQ ID NO: 3). In some embodiments, BCMA proteins include murine BCMA (SEQ ID NO: 4).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (V_{H} or VH) and a heavy chain constant region (CdH or CH). The heavy chain constant region typically comprises three domains, abbreviated C_{H}1 (or CH1), C_{H}2 (or CH2), and C_{H}3 (or CH3). Each light chain typically comprises a light chain variable region (V_{L} or VL) and a light chain constant region. The light chain constant region typically comprises one domain, abbreviated C_{L} or CL.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. A “BCMA antibody,” “anti-BCMA antibody,” “BCMA Ab,” “BCMA-specific antibody,” “anti-BCMA Ab,” “BCMA antibody,” “anti-BCMA antibody,” “BCMA Ab,” “BCMA-specific antibody,” or “anti-BCMA Ab,” or any iteration of these phrases where “BCMA” is substituted by “TNFSF17,” is an antibody, as described herein, which binds specifically to BCMA. In some embodiments, the antibody binds the extracellular domain of BCMA.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, Md., incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J Mol. Biol., 273:927-948 (“Chothia” numbering scheme); MacCallum et al., 1996, J Mol. Biol. 262:732-745 (“Contact” numbering scheme); Lefranc et al., Dev. Comp. Immunol., 2003, 27:55-77 (“IMGT” numbering scheme); and Honegge and Plückthun, I Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE 1
Residues in CDRs according to
Kabat and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 | H31-H35B | H26-H32 or H34* |
| (Kabat Numbering) | | |
| H1 | H31-H35 | H26-H32 |
| (Chothia Numbering) | | |
| H2 | H50-H65 | H52-H56 |
| H3 | H95-H102 | H95-H102 |

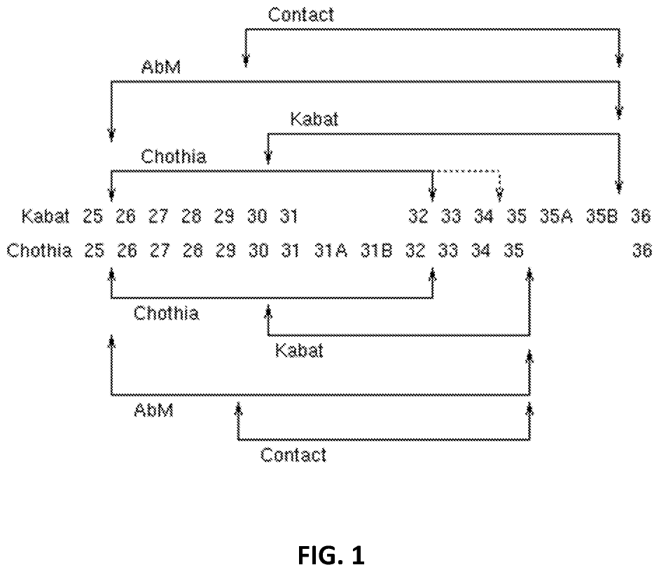

*The C-terminus of CDR-H1, when numbered using the Kabat numbering convention, varies between H32 and H34, depending on the length of the CDR, as illustrated in FIG. 1.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (CFH) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is SEQ ID NO: 26. In some embodiments, the linker is SEQ ID NO: 27. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain. In some embodiments, the IgG1 Fc domain comprises SEQ ID NO: 19, or a portion thereof. SEQ ID NO: 19 provides the sequence of C_{H}1, C_{H}2, and C_{H}3 of the human IgG1 constant region.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” or “kd” (sec⁻¹), as used herein, refers to the dissociation rate constant of a particular antibody-antigen interaction. This value is also referred to as the k_{off} value.

The term “kₐ” or “ka” (M⁻¹×sec⁻¹), as used herein, refers to the association rate constant of a particular antibody-antigen interaction. This value is also referred to as the kₒₙ value.

The term “K_{D}” (also referred to as “Kd” or “KD,” M or nM), as used herein, refers to the dissociation equilibrium constant of a particular antibody-antigen interaction. K_{D}=k_{d}/kₐ. The value of K_{D} is typically equal in magnitude to the concentration of ligand at which half the protein molecules are bound to ligand at equilibrium.

The term “K_{A}” or “Kₐ” (M⁻¹), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and VL domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., BCMA). In one exemplary assay, BCMA is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind the antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to variants of BCMA with different point-mutations.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. Polypeptide sequences having such substitutions are known as “conservatively modified variants.” By way of example, the groups of amino acids provided in Tables 2-4 are, in some embodiments, considered conservative substitutions for one another.

TABLE 2
Selected groups of amino acids that
are considered conservative substitutions
for one another, in certain embodiments.

| | |
|---|---|
| Acidic Residues | D and E |
| Basic Residues | K, R, and H |
| Hydrophilic | S, T, N, and Q |
| Uncharged Residues | |
| Aliphatic | G, A, V, L, and I |
| Uncharged Residues | |
| Non-polar | C, M, and P |
| Uncharged Residues | |
| Aromatic Residues | F, Y, and W |
| Alcohol Group- | S and T |
| Containing Residues | |
| Aliphatic Residues | I, L, V, and M |
| Cycloalkenyl- | F, H, W, and Y |
| associated Residues | |
| Hydrophobic | A, C, F, G, H, I, L, |
| Residues | M, R, T, V, W, and Y |
| Negatively | D and E |
| Charged Residues | |
| Polar Residues | C, D, E, H, K, |
| | N, Q, R, S, and T |
| Positively | H, K, and R |
| Charged Residues | |
| Small Residues | A, C, D, G, N, |
| | P, S, T, and V |
| Very Small Residues | A, G, and S |
| Residues Involved | A, C, D, E, G, H, K, |
| in Turn Formation | N, Q, R, S, P, and T |
| Flexible Residues | Q, T, K, S, G, P, D, E, and R |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that
are considered conservative substitutions
for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Naturally encoded amino acids are the proteinogenic amino acids known to those of skill in the art. They include the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and the less common pyrrolysine and selenocysteine. Naturally encoded amino acids include post-translational variants of the 22 naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids and acylated amino acids.

The term “non-natural amino acid” refers to an amino acid that is not a proteinogenic amino acid, or a post-translationally modified variant thereof. In particular, the term refers to an amino acid that is not one of the 20 common amino acids or pyrrolysine or selenocysteine, or post-translationally modified variants thereof.

The term “conjugate” or “antibody conjugate” refers to an antibody linked to one or more payload moieties. The antibody can be any antibody described herein. The payload can be any payload described herein. The antibody can be directly linked to the payload via a covalent bond, or the antibody can be linked to the payload indirectly via a linker. Typically, the linker is covalently bonded to the antibody and also covalently bonded to the payload. The term “antibody drug conjugate” or “ADC” refers to a conjugate wherein at least one payload is a therapeutic moiety such as a drug.

The term “payload” refers to a molecular moiety that can be conjugated to an antibody. In particular embodiments, payloads are selected from the group consisting of therapeutic moieties and labelling moieties.

The term “linker” refers to a molecular moiety that is capable of forming at least two covalent bonds. Typically, a linker is capable of forming at least one covalent bond to an antibody and at least another covalent bond to a payload. In certain embodiments, a linker can form more than one covalent bond to an antibody. In certain embodiments, a linker can form more than one covalent bond to a payload or can form covalent bonds to more than one payload. After a linker forms a bond to an antibody, or a payload, or both, the remaining structure, i.e. the residue of the linker after one or more covalent bonds are formed, may still be referred to as a “linker” herein. The term “linker precursor” refers to a linker having one or more reactive groups capable of forming a covalent bond with an antibody or payload, or both. In some embodiments, the linker is a cleavable linker. For example, a cleavable linker can be one that is released by an bio-labile function, which may or may not be engineered. In some embodiments, the linker is a non-cleavable linker. For example, a non-cleavable linker can be one that is released upon degradation of the antibody.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder. In some embodiments, a therapeutically effective amount or effective amount refers to an amount of an antibody or composition that when administered to a subject is effective to prevent or ameliorate a disease or the progression of the disease, or result in amelioration of symptoms.

As used herein, the term “inhibits growth” (e.g. referring to cells, such as tumor cells) is intended to include any measurable decrease in cell growth (e.g., tumor cell growth) when contacted with a BCMA antibody, as compared to the growth of the same cells not in contact with a BCMA antibody. In some embodiments, growth may be inhibited by at least about 10%, 20%, 30%, 40%, 50%, 60%, 70%, 80%, 90%, 99%, or 100%. The decrease in cell growth can occur by a variety of mechanisms, including but not limited to antibody internalization, apoptosis, necrosis, and/or effector function-mediated activity.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a disease that can be treated or diagnosed with an antibody provided herein. In some embodiments, the disease is leukemia, lymphoma, or multiple myeloma, a plasmacytoid dendritic cell tumor, a B-cell lineage malignancy, a plasma cell neoplasm, diffuse large B-cell lymophoma (DLBCL), a low-grade B-cell lymphoma, Burkitt's lymphoma, a plasmablastic lymphoma, or a follicular lymphoma.

In some chemical structures illustrated herein, certain substituents, chemical groups, and atoms are depicted with a curvy/wavy line (e.g., [symbol] ) that intersects a bond or bonds to indicate the atom through which the substituents, chemical groups, and atoms are bonded. For example, in some structures, such as but not limited to

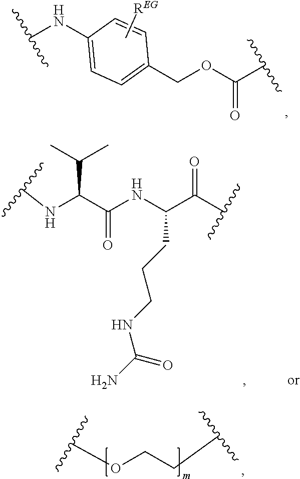

this curvy/wavy line indicates the atoms in the backbone of a conjugate or linker-payload structure to which the illustrated chemical entity is bonded. In some structures, such as but not limited to

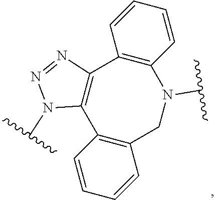 [SMILES: *N1Cc2ccccc2-c2c(nnn2*)-c2ccccc21]

this curvy/wavy line indicates the atoms in the antibody or antibody fragment as well as the atoms in the backbone of a conjugate or linker-payload structure to which the illustrated chemical entity is bonded.

The term “site-specific” refers to a modification of a polypeptide at a predetermined sequence location in the polypeptide. The modification is at a single, predictable residue of the polypeptide with little or no variation. In particular embodiments, a modified amino acid is introduced at that sequence location, for instance recombinantly or synthetically. Similarly, a moiety can be “site-specifically” linked to a residue at a particular sequence location in the polypeptide. In certain embodiments, a polypeptide can comprise more than one site-specific modification.

### 2. Conjugates

Provided herein are conjugates of antibodies to BCMA. The conjugates comprise an antibody to BCMA covalently linked via a linker to a payload. In certain embodiments, the antibody is linked to one payload. In further embodiments, the antibody is linked to more than one payload. In certain embodiments, the antibody is linked to two, three, four, five, six, seven, eight, or more payloads.

In the conjugates provided herein, the antibody can be from any species. In certain embodiments, the BCMA is a vertebrate BCMA. In certain embodiments, the BCMA is a mammalian BCMA. In certain embodiments, the BCMA is human BCMA. In certain embodiments, the BCMA is mouse BCMA. In certain embodiments, the BCMA is cynomolgus BCMA.

The antibody is typically a protein comprising multiple polypeptide chains. In certain embodiments, the antibody is a heterotetramer comprising two identical light (L) chains and two identical heavy (H) chains. Each light chain can be linked to a heavy chain by one covalent disulfide bond. Each heavy chain can be linked to the other heavy chain by one or more covalent disulfide bonds. Each heavy chain and each light chain can also have one or more intrachain disulfide bonds. As is known to those of skill in the art, each heavy chain typically comprises a variable domain (VH) followed by a number of constant domains. Each light chain typically comprises a variable domain at one end (V_{L}) and a constant domain. As is known to those of skill in the art, antibodies typically have selective affinity for their target molecules, i.e. antigens.

The antibodies provided herein can have any antibody form known to those of skill in the art. They can be full-length, or fragments. Exemplary full length antibodies include IgA, IgA1, IgA2, IgD, IgE, IgG, IgG1, IgG2, IgG3, IgG4, IgM, etc. Exemplary fragments include Fv, Fab, Fc, scFv, scFv-Fc, etc.

In certain embodiments, the antibody of the conjugate comprises six of the CDR sequences described herein. In certain embodiments, the antibody of the conjugate comprises a heavy chain variable domain (V_{H}) described herein. In certain embodiments, the antibody of the conjugate comprises a light chain variable domain (V_{L}) described herein. In certain embodiments, the antibody of the conjugate comprises a heavy chain variable domain (V_{H}) described herein and a light chain variable domain (V_{L}) described herein. In certain embodiments, the antibody of the conjugate comprises a paired heavy chain variable domain and a light chain variable domain described herein (V_{H}-V_{L} pair).

In certain embodiments, the antibody conjugate can be formed from an antibody that comprises one or more reactive groups. In certain embodiments, the antibody conjugate can be formed from an antibody comprising all naturally encoded amino acids. Those of skill in the art will recognize that several naturally encoded amino acids include reactive groups capable of conjugation to a payload or to a linker. These reactive groups include cysteine side chains, lysine side chains, and amino-terminal groups. In these embodiments, the antibody conjugate can comprise a payload or linker linked to the residue of an antibody reactive group. In these embodiments, the payload precursor or linker precursor comprises a reactive group capable of forming a bond with an antibody reactive group. Typical reactive groups include maleimide groups, activated carbonates (including but not limited to, p-nitrophenyl ester), activated esters (including but not limited to, N-hydroxysuccinimide, p-nitrophenyl ester, and aldehydes). Particularly useful reactive groups include maleimide and succinimide, for instance N-hydroxysuccinimide, for forming bonds to cysteine and lysine side chains. Further reactive groups are described in the sections and examples below.

In further embodiments, the antibody comprises one or more modified amino acids having a reactive group, as described herein. Typically, the modified amino acid is not a naturally encoded amino acid. These modified amino acids can comprise a reactive group useful for forming a covalent bond to a linker precursor or to a payload precursor. One of skill in the art can use the reactive group to link the polypeptide to any molecular entity capable of forming a covalent bond to the modified amino acid. Thus, provided herein are conjugates comprising an antibody comprising a modified amino acid residue linked to a payload directly or indirectly via a linker. Exemplary modified amino acids are described in the sections below. Generally, the modified amino acids have reactive groups capable of forming bonds to linkers or payloads with complementary reactive groups.

In certain embodiments, the non-natural amino acids are positioned at select locations in a polypeptide chain of the antibody. These locations were identified as providing optimum sites for substitution with the non-natural amino acids. Each site is capable of bearing a non-natural amino acid with optimum structure, function and/or methods for producing the antibody.

In certain embodiments, a site-specific position for substitution provides an antibody that is stable. Stability can be measured by any technique apparent to those of skill in the art.

In certain embodiments, a site-specific position for substitution provides an antibody that has optimal functional properties. For instance, the antibody can show little or no loss of binding affinity for its target antigen compared to an antibody without the site-specific non-natural amino acid. In certain embodiments, the antibody can show enhanced binding compared to an antibody without the site-specific non-natural amino acid.

In certain embodiments, a site-specific position for substitution provides an antibody that can be made advantageously. For instance, in certain embodiments, the antibody shows advantageous properties in its methods of synthesis, discussed below. In certain embodiments, the antibody can show little or no loss in yield in production compared to an antibody without the site-specific non-natural amino acid. In certain embodiments, the antibody can show enhanced yield in production compared to an antibody without the site-specific non-natural amino acid. In certain embodiments, the antibody can show little or no loss of tRNA suppression compared to an antibody without the site-specific non-natural amino acid. In certain embodiments, the antibody can show enhanced tRNA suppression in production compared to an antibody without the site-specific non-natural amino acid.

In certain embodiments, a site-specific position for substitution provides an antibody that has advantageous solubility. In certain embodiments, the antibody can show little or no loss in solubility compared to an antibody without the site-specific non-natural amino acid. In certain embodiments, the antibody can show enhanced solubility compared to an antibody without the site-specific non-natural amino acid.

In certain embodiments, a site-specific position for substitution provides an antibody that has advantageous expression. In certain embodiments, the antibody can show little or no loss in expression compared to an antibody without the site-specific non-natural amino acid. In certain embodiments, the antibody can show enhanced expression compared to an antibody without the site-specific non-natural amino acid.

In certain embodiments, a site-specific position for substitution provides an antibody that has advantageous folding. In certain embodiments, the antibody can show little or no loss in proper folding compared to an antibody without the site-specific non-natural amino acid. In certain embodiments, the antibody can show enhanced folding compared to an antibody without the site-specific non-natural amino acid.

In certain embodiments, a site-specific position for substitution provides an antibody that is capable of advantageous conjugation. As described below, several non-natural amino acids have side chains or functional groups that facilitate conjugation of the antibody to a second agent, either directly or via a linker. In certain embodiments, the antibody can show enhanced conjugation efficiency compared to an antibody without the same or other non-natural amino acids at other positions. In certain embodiments, the antibody can show enhanced conjugation yield compared to an antibody without the same or other non-natural amino acids at other positions. In certain embodiments, the antibody can show enhanced conjugation specificity compared to an antibody without the same or other non-natural amino acids at other positions.

The one or more non-natural amino acids are located at selected site-specific positions in at least one polypeptide chain of the antibody. The polypeptide chain can be any polypeptide chain of the antibody without limitation, including either light chain or either heavy chain. The site-specific position can be in any domain of the antibody, including any variable domain and any constant domain.

In certain embodiments, the antibodies provided herein comprise one non-natural amino acid at a site-specific position. In certain embodiments, the antibodies provided herein comprise two non-natural amino acids at site-specific positions. In certain embodiments, the antibodies provided herein comprise three non-natural amino acids at site-specific positions. In certain embodiments, the antibodies provided herein comprise more than three non-natural amino acids at site-specific positions.

In certain embodiments, the antibodies provided herein comprise non-natural amino acids each at the positions HC-F404 and HC-Y180, according to the Kabat or Chothia or EU numbering scheme, or a post-translationally modified variant thereof. In these designations, HC indicates a heavy chain residue, and LC indicates a light chain residue. Those of skill will recognize tht the non-natural amino acids substitute for the residues HC-F404 and HC-Y180 in the antibody amino acid sequence. In certain embodiments, the non-natural amino acids are residues of Formula (30), herein.

### 3. Conjugating Groups and Residues Thereof

Conjugating groups facilitate conjugation of the payloads described herein to a second compound, such as an antibody described herein. In certain embodiments, the conjugating group is designated R herein. Conjugating groups can react via any suitable reaction mechanism known to those of skill in the art. In certain embodiments, a conjugating group reacts through a [3+2] alkyne-azide cycloaddition reaction, inverse-electron demand Diels-Alder ligation reaction, thiol-electrophile reaction, or carbonyl-oxyamine reaction, as described in detail herein. In certain embodiments, the conjugating group comprises an alkyne, for instance a strained alkyne. In certain embodiments, the conjugating group is:

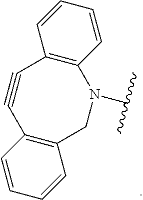 [SMILES: *N1Cc2ccccc2C#Cc2ccccc21]

Additional conjugating groups are described in, for example, U.S. Patent Publication No. 2014/0356385, U.S. Patent Publication No. 2013/0189287, U.S. Patent Publication No. 2013/0251783, U.S. Pat. Nos. 8,703,936, 9,145,361, 9,222,940, and 8,431,558.

After conjugation, a divalent residue of the conjugating group is formed and is bonded to the residue of a second compound. The structure of the divalent residue is determined by the type of conjugation reaction employed to form the conjugate.

In certain embodiments when a conjugate is formed through a [3+2] alkyne-azide cycloaddition reaction, the divalent residue of the conjugating group comprises a triazole ring or fused cyclic group comprising a triazole ring. In certain embodiments when a conjugate is formed through a strain-promoted [3+2] alkyne-azide cycloaddition (SPAAC) reaction, the divalent residue of the conjugating group is:

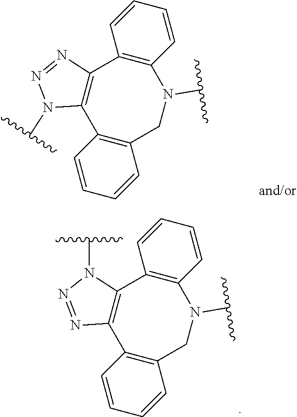 [SMILES: *N1Cc2ccccc2-c2c(nnn2*)-c2ccccc21 ; *N1Cc2ccccc2-c2nnn(*)c2-c2ccccc21]

In an embodiment, provided herein is a conjugate according to any of Formulas 101a-105b, where COMP indicates a residue of the anti-BCMA antibody and PAY indicates the payload moiety:

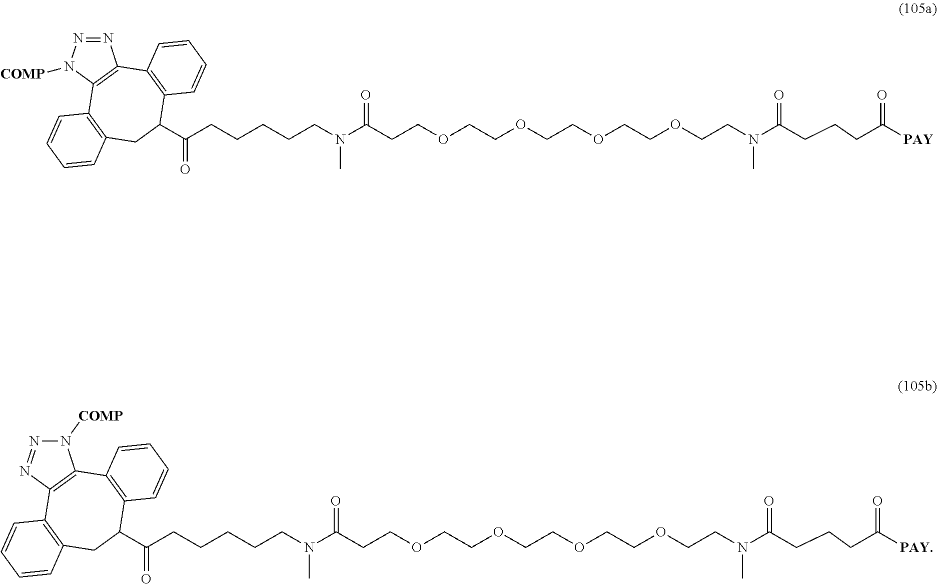

In any of the foregoing embodiments, the conjugate comprises n number of PAY moieties, wherein n is an integer from 1 to 8. In some embodiments, n is 2. In some embodiments, n is 3. In some embodiments, n is 4. In some embodiments, n is 5. In some embodiments, n is 6. In some embodiments, n is 7. In some embodiments, n is 8.

In particular embodiments, provided herein are anti-BCMA conjugates according to any of Formulas 105a-105b wherein COMP indicates a residue of the non-natural amino acid according to Formula (30), below. In particular embodiments, provided herein are anti-BCMA conjugates according to any of Formulas 105a-105b wherein COMP indicates a residue of the non-natural amino acid according to Formula (30), below, at heavy chain position 404 according to the EU numbering system. In particular embodiments, provided herein are anti-BCMA conjugates according to any of Formulas 105a-105b wherein COMP indicates a residue of the non-natural amino acid according to Formula (30), below, at heavy chain position 180 according to the EU numbering system.

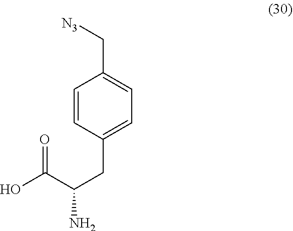 [SMILES: [N-]=[N+]=NCc1ccc(C[C@H](N)C(=O)O)cc1]

Those of skill will recognize that amino acids such as Formula (30) are incorporated into polypeptides and antibodies as residues. For instance, a residue of Formula (30) can be according to the following Formula:

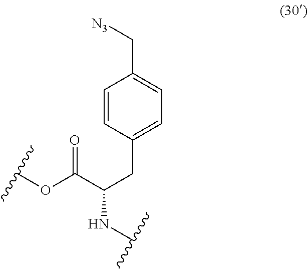 [SMILES: *N[C@@H](Cc1ccc(CN=[N+]=[N-])cc1)C(=O)O*]

Further modification, for instance at —N₃ is also encompassed within the term residue herein.

In an embodiment, provided herein is a conjugate according to any of Formulas 105c-105d, where COMP indicates a residue of the anti-BCMA antibody and PAY indicates the payload moiety:

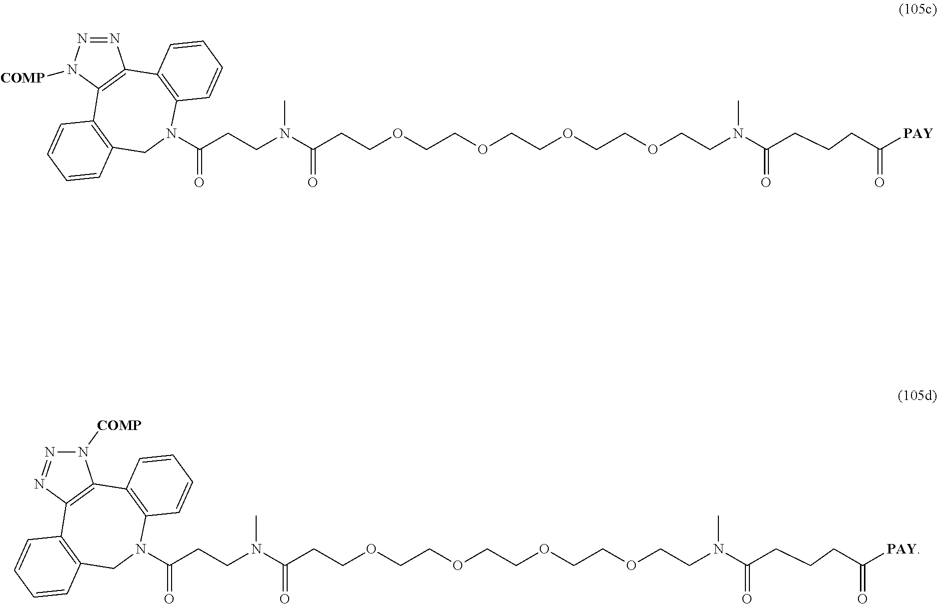

In any of the foregoing embodiments, the conjugate comprises n number of PAY moieties, wherein n is an integer from 1 to 8. In some embodiments, n is 2. In some embodiments, n is 3. In some embodiments, n is 4. In some embodiments, n is 5. In some embodiments, n is 6. In some embodiments, n is 7. In some embodiments, n is 8.

In particular embodiments, provided herein are anti-BCMA conjugates according to any of Formulas 105c-105d wherein COMP indicates a residue of the non-natural amino acid according to Formula (30), below. In particular embodiments, provided herein are anti-BCMA conjugates according to any of Formulas 105c-105d wherein COMP indicates a residue of the non-natural amino acid according to Formula (30), below, at heavy chain position 404 according to the EU numbering system. In particular embodiments, provided herein are anti-BCMA conjugates according to any of Formulas 105c-105d wherein COMP indicates a residue of the non-natural amino acid according to Formula (30), below, at heavy chain position 180 according to the EU numbering system.

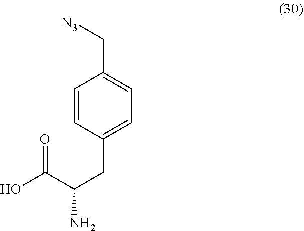 [SMILES: [N-]=[N+]=NCc1ccc(C[C@H](N)C(=O)O)cc1]

Those of skill will recognize that amino acids such as Formula (30) are incorporated into polypeptides and antibodies as residues. For instance, a residue of Formula (30) can be according to the following Formula:

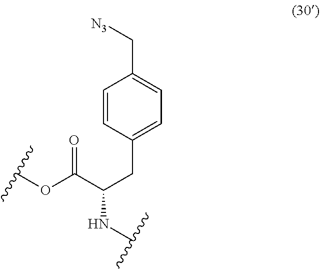 [SMILES: *N[C@@H](Cc1ccc(CN=[N+]=[N-])cc1)C(=O)O*]

Further modification, for instance at —N3 is also encompassed within the term residue herein.

In particular embodiments, provided herein are anti-BCMA conjugates having the structure of Conjugate M:

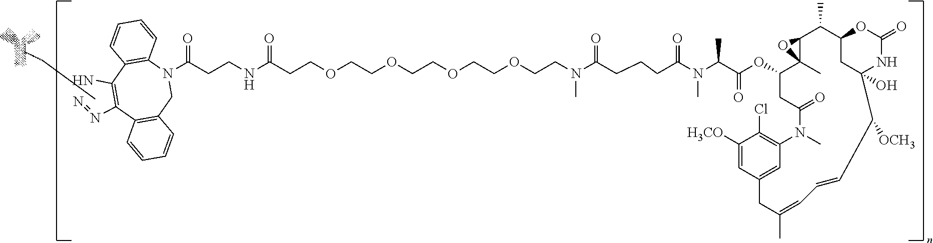

where n is an integer from 1 to 6. In some embodiments, n is an integer from 1 to 4. In some embodiments, n is 2. For example, in particular embodiments, the anti-BCMA conjugate has the structure:

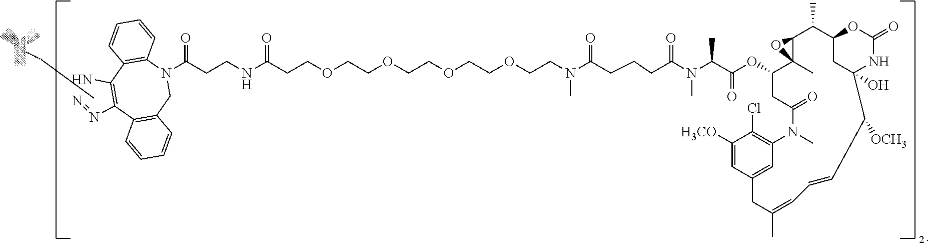

In some embodiments, n is 4. For example, in particular embodiments, the anti-BCMA conjugate has the structure:

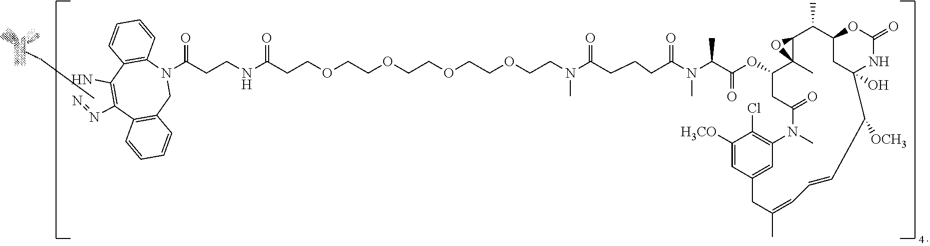

In any of the foregoing embodiments wherein the anti-BCMA conjugate has a structure according to Conjugate M, the bracketed structure can be covalently bonded to one or more non-natural amino acids of the antibody at sites HC-F404 and HC-Y180, according to the Kabat or EU numbering scheme of Kabat. In particular embodiments, each non-natural amino acid is a residue according to Formula (30).

In one embodiment, the anti-BCMA conjugate is Conjugate 4, having the structure of:

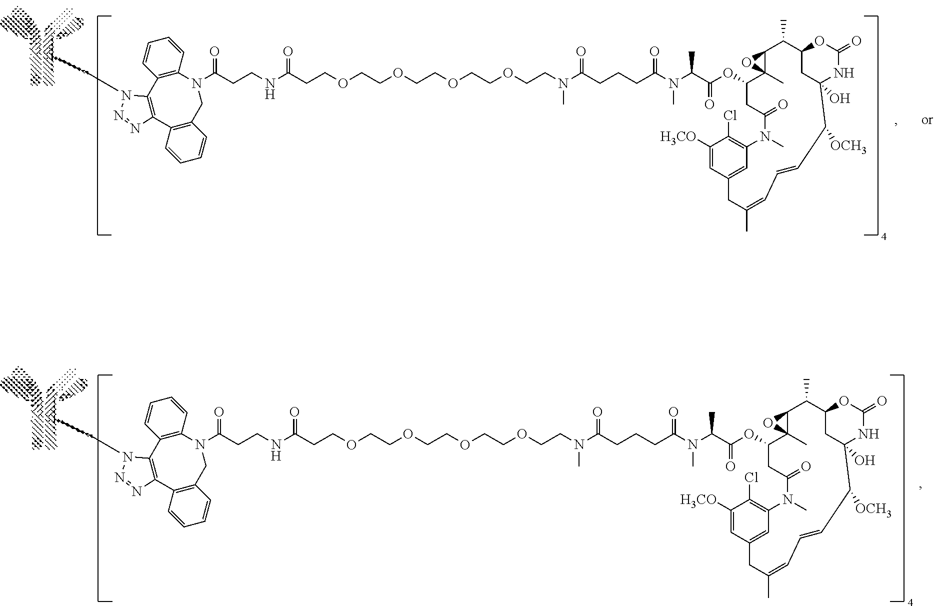

wherein the antibody comprises a heavy chain sequence provided in SEQ ID NO: 15, and a light chain sequence provided in SEQ ID NO: 17;
wherein the antibody further comprises residues of p-azidomethyl-phenylalanine substituting at each of sites HC-F404 and HC-Y180 according to the EU numbering scheme; and
each structure within the brackets of the formulas is bonded to the antibody at one of the p-azidomethyl-phenylalanine residues.

In one embodiment, the anti-BCMA conjugate is Conjugate 4, wherein the predominant species is:

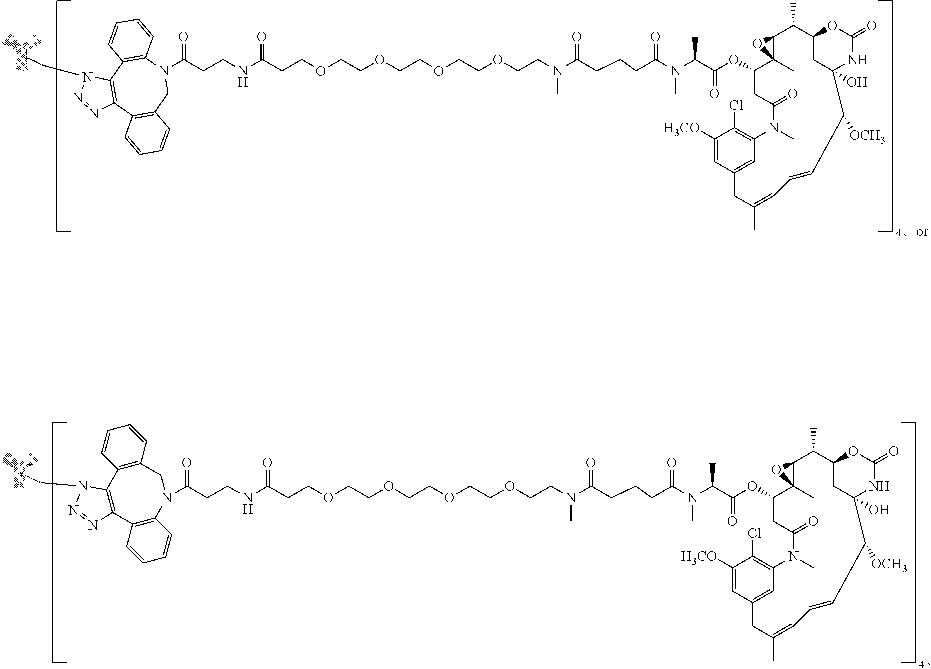

wherein the antibody comprises a heavy chain sequence provided in SEQ ID NO: 15, and a light chain sequence provided in SEQ ID NO: 17;
wherein the antibody further comprises residues of p-azidomethyl-phenylalanine substituting at each of sites HC-F404 and HC-Y180 according to the EU numbering scheme; and
each structure within the brackets of the formulas is bonded to the antibody at one of the p-azidomethyl-phenylalanine residues.

In one embodiment, the anti-BCMA conjugate is Conjugate 4, wherein the predominant species is:

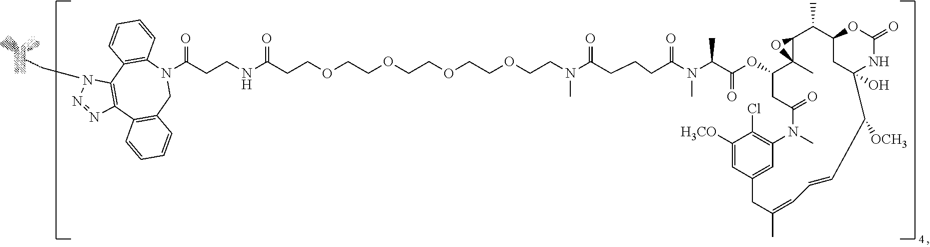

wherein the antibody comprises a heavy chain sequence provided in SEQ ID NO: 15, and a light chain sequence provided in SEQ ID NO: 17;
wherein the antibody further comprises residues of p-azidomethyl-phenylalanine substituting at each of sites HC-F404 and HC-Y180 according to the EU numbering scheme; and
each structure within the brackets of the formulas is bonded to the antibody at one of the p-azidomethyl-phenylalanine residues.

In one embodiment, the anti-BCMA conjugate is Conjugate 4, wherein the predominant species is:

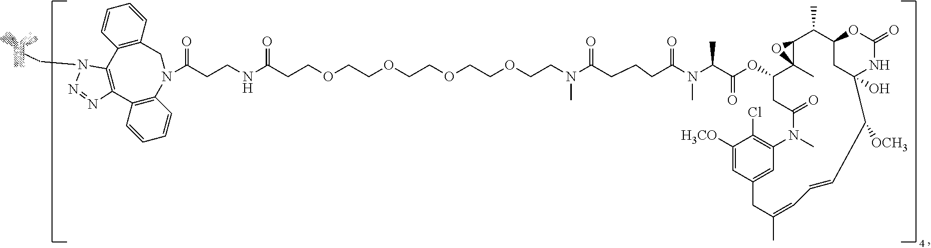

wherein the antibody comprises a heavy chain sequence provided in SEQ ID NO: 15, and a light chain sequence provided in SEQ ID NO: 17;
wherein the antibody further comprises residues of p-azidomethyl-phenylalanine substituting at each of sites HC-F404 and HC-Y180 according to the EU numbering scheme; and
each structure within the brackets of the formulas is bonded to the antibody at one of the p-azidomethyl-phenylalanine residues.

### 4. Antibody Specificity

The conjugates comprise antibodies that selectively bind human BCMA. In some aspects, the antibody selectively binds to the extracellular domain of human BCMA (human BCMA).

In some embodiments, the antibody binds to a homolog of human BCMA. In some aspects, the antibody binds to a homolog of human BCMA from a species selected from monkeys, mice, dogs, cats, rats, cows, horses, goats and sheep. In some aspects, the homolog is a cynomolgus monkey homolog. In some aspects, the homolog is a mouse or murine homolog.

In some embodiments, the antibody comprises a light chain. In some aspects, the light chain is a kappa light chain. In some aspects, the light chain is a lambda light chain. In specific embodiments, the kappa light chain comprises a constant region comprising the amino acid sequence provided SEQ ID NO: 20.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

The antibody conjugates provided herein may be useful for the treatment of a variety of diseases and conditions including cancers. In some embodiments, the antibody conjugates provided herein may be useful for the treatment of cancers of solid tumors. For example, the antibody conjugates provided herein can be useful for the treatment of colorectal cancer.

In some embodiments, the antibody comprises, consists of, or consists essentially of a V_{H} sequence provided in SEQ ID NO: 13. In some embodiments, the antibody comprises, consists of, or consists essentially of a V_{L} sequence provided in SEQ ID NO: 14. In some embodiments, the antibody comprises a V_{H} sequence and a V_{L} sequence. In some aspects, the V_{H} sequence is a V_{H} sequence comprising, consisting of, or consisting essentially of any one of SEQ ID NO: 13, and the V_{L} sequence is a V_{L} sequence comprising, consisting of, or consisting essentially of any one of SEQ ID NO: 14. In certain embodiments, the antibody comprises, consists of, or consists essentially of, a heavy chain sequence provided in SEQ ID NO: 15. In a specific embodiments, the heavy chain sequence, e.g., heavy chain sequence provided in SEQ ID NO: 15, additionally comprises an N-terminal methionine. An certain embodiments, such heavy chain sequence is encoded by the nucleotide sequence provided in SEQ ID NO: 16. In certain embodiments, the antibody comprises, consists of, or consists essentially of, a light chain sequence provided in SEQ ID NO: 17. In a specific embodiments, the light chain sequence, e.g., light chain sequence provided in SEQ ID NO: 17, additionally comprises an N-terminal methionine. An certain embodiments, such light chain sequence is encoded by the nucleotide sequence provided in SEQ ID NO: 18.

In some embodiments, the antibodies comprise six of the CDRs indicated in Table 5 below. In particular embodiments, Chothia CDRs are selected. In particular embodiments, Kabat CDRs are selected.

TABLE 5
Antibody 2265-F02 CDRs.

| | Chothia | Kabat | Chothia | Kabat | | | | |
|---|---|---|---|---|---|---|---|---|
| | CDR | CDR | CDR | CDR | CDR | CDR | CDR | CDR |
| | H1 | H1 | H2 | H2 | H3 | L1 | L2 | L3 |
| | SEQ | SEQ | SEQ | SEQ | SEQ | SEQ | SEQ | SEQ |
| | ID | ID | ID | ID | ID | ID | ID | ID |
| | NO | NO | NO | NO | NO | NO | NO | NO |
| 2265-F02 | 5 | 6 | 7 | 8 | 9 | 10 | 11 | 12 |

In some embodiments, the antibody comprises three of: a CDR-H1 comprising one of SEQ ID NOs: 5 and 6; a CDR-H2 comprising one of SEQ ID NOs: 7 and 8; a CDR-H3 comprising SEQ ID NO: 9; and one, two, or all three of: a CDR-L1 comprising SEQ ID NO: 10; a CDR-L2 comprising SEQ ID NO: 11; and a CDR-L3 comprising SEQ ID NO: 12. In particular embodiments, the CDRs are according to Chothia. In particular embodiments, the CDRs are according to Kabat.

### 5. Germline

In some embodiments, the antibody that specifically binds BCMA is an antibody comprising a variable region that is encoded by a particular germline gene, or a variant thereof. The illustrative antibodies provided herein comprise variable regions that are encoded by the heavy chain variable region germline genes VH1-18, VH3-33, VH2-5, VH2-70, and VH4-30-4. or variants thereof; and the light chain variable region germline genes Vκ1-5, Vκ3-11, Vκ2-20, Vκ1-33, and Vκ1-16, or variants thereof.

One of skill in the art would recognize that the CDR sequences provided herein may also be useful when combined with variable regions encoded by other variable region germline genes, or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the V_{H} 1, V_{H} 2, V_{H} 3, or V_{H} 4 families, or a variant thereof. In some embodiments, a CDR-L sequence provided herein may be combined with a variable region encoded by a variable region germline gene selected from the Vκ1, Vκ2, or Vκ3, or a variant thereof.

### 6. Glycosylation Variants

In certain embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

### 7. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In some embodiments, the Fc comprises one or more modifications in at least one of the C_{H}3 sequences. In some embodiments, the Fc comprises one or more modifications in at least one of the C_{H}2 sequences. For example, the Fc can include one or modifications selected from the group consisting of: V262E, V262D, V262K, V262R, V262S, V264S, V303R, and V305R. In some embodiments, an Fc is a single polypeptide. In some embodiments, an Fc is multiple peptides, e.g., two polypeptides. Exemplary modifications in the Fc region are described, for example, in International Patent Application No. PCT/US2017/037545, filed Jun. 14, 2017.

An alteration in in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγRI, FcγRII and FcγRIII FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

FcRn binding and in vivo clearance (half-life determination) can also be measured, for example, using the methods described in Petkova et al., Intl. Immunol., 2006, 18:1759-1769, incorporated by reference in its entirety.

### 8. Modified Amino Acids

When the antibody conjugate comprises a modified amino acid, the modified amino acid can be any modified amino acid deemed suitable by the practitioner. In particular embodiments, the modified amino acid is p-azido-methyl-L-phenylalanine (also referred to as p-methylazido phenylalanine). In particular embodiments, the non-natural amino acid is compound (30):

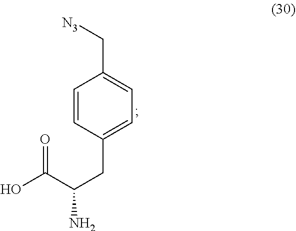 [SMILES: [N-]=[N+]=NCc1ccc(C[C@H](N)C(=O)O)cc1]

or a salt thereof. Such non-natural amino acids may be in the form of a salt. It will be understood by one of ordinary skill in the art that the azido moiety of the p-azido-methyl-L-phenylalanine residue reacts with a conjugating group to form the triazole of the fused cyclic group formed through the strain-promoted [3+2] alkyne-azide cycloaddition reaction used to make certain of the conjugates described herein.

### 9. Preparation of Antibody Conjugates

9.1. Antigen Preparation

The BCMA protein to be used for isolation of the antibodies may be intact BCMA or a fragment of BCMA. The intact BCMA protein, or fragment of BCMA, may be in the form of an isolated protein or protein expressed by a cell. Other forms of BCMA useful for generating antibodies will be apparent to those skilled in the art.

9.2. Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, Calif., incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, Calif.), and SP-2 or X63-Ag8-653 cells (available from the American Type Culture Collection, Rockville, Md.). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

9.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

9.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

9.5. Conjugation

The antibody conjugates can be prepared by standard techniques. In certain embodiments, an antibody is contacted with a payload precursor under conditions suitable for forming a bond from the antibody to the payload to form an antibody-payload conjugate. In certain embodiments, an antibody is contacted with a linker precursor under conditions suitable for forming a bond from the antibody to the linker. The resulting antibody-linker is contacted with a payload precursor under conditions suitable for forming a bond from the antibody-linker to the payload to form an antibody-linker-payload conjugate. In certain embodiments, a payload precursor is contacted with a linker precursor under conditions suitable for forming a bond from the payload to the linker. The resulting payload-linker is contacted with an antibody under conditions suitable for forming a bond from the payload-linker to the antibody to form an antibody-linker-payload conjugate. Suitable linkers for preparing the antibody conjugates are disclosed herein, and exemplary conditions for conjugation are described in the Examples below.

In some embodiments, an anti-BCMA conjugate is prepared by contacting an anti-BCMA antibody as disclosed herein with a linker precursor having a structure (M):

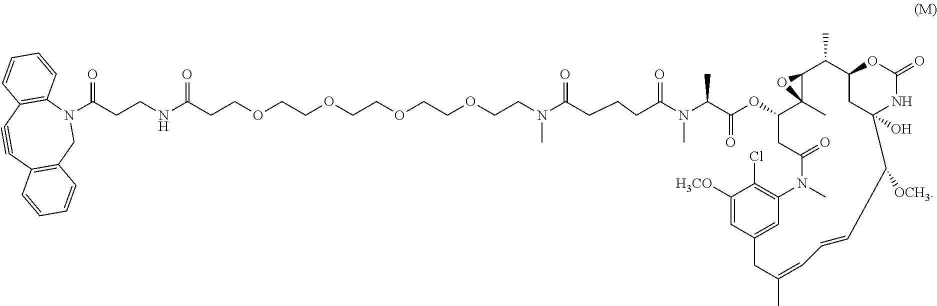 [SMILES: COc1cc2cc(c1Cl)N(C)C(=O)C[C@H](OC(=O)[C@H](C)N(C)C(=O)CCCC(=O)N(C)CCOCCOCCOCCOCCC(=O)NCCC(=O)N1Cc3ccccc3C#Cc3ccccc31)[C@]1(C)O[C@H]1[C@H](C)[C@@H]1C[C@@](O)(NC(=O)O1)[C@H](OC)/C=C/C=C(/C)C2]

Such a linker precursor can be prepared by standard techniques, or obtained from commercial sources, e.g. WO 2019/055931, WO 2019/055909, WO 2017/132617, WO 2017/132615, each incorporated by reference in its entirety.

It will be understood that the conjugates from the conjugation reaction disclosed herein may result in a mixture of conjugates with a distribution of one or more drugs (e.g., PAY moieties) attached to an antibody. Individual conjugates may be identified in the mixture by, for example, mass spectroscopy and separated by HPLC, e.g., hydrophobic interaction chromatography, including such methods known in the art. In certain embodiments, the mixture of conjugates comprises a predominant conjugate species. In certain embodiments, a homogeneous conjugate with a single drug to antibody ratio (DAR) value may be isolated from the conjugation mixture, for example by electrophoresis or chromatography.

DAR may range from 1 to 8 units per conjugate. The quantitative distribution of DAR in terms of n may also be determined. In some instances, separation, purification, and characterization of homogeneous conjugate where n is a certain value may be achieved by means such as electrophoresis.

In certain embodiments, the DAR for a conjugate provided herein ranges from 1 to 8. In certain embodiments, the DAR for a conjugate provided herein ranges from about 2 to about 6; from about 3 to about 5.

In some embodiments, the DAR for a conjugate provided herein is about 1. In some embodiments, the DAR for a conjugate provided herein is about 2. In some embodiments, the DAR for a conjugate provided herein is about 2.5. In some embodiments, the DAR for a conjugate provided herein is about 3. In some embodiments, the DAR for a conjugate provided herein is about 3.5. In some embodiments, the DAR for a conjugate provided herein is about 4. In some embodiments, the DAR for a conjugate provided herein is about 3.0, about 3.1, about 3.2, about 3.3, about 3.4, about 3.5, about 3.6, about 3.7, about 3.8, or about 3.9. In some embodiments, the DAR for a conjugate provided herein is about 5. In some embodiments, the DAR for a conjugate provided herein is about 6. In some embodiments, the DAR for a conjugate provided herein is about 7. In some embodiments, the DAR for a conjugate provided herein is about 8.

In some preferred embodiments, the DAR for a conjugate provided herein is about 4.

### 10. Vectors, Host Cells, and Recombinant Methods

Embodiments are also directed to the provision of isolated nucleic acids encoding anti-BCMA antibodies, vectors and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Envinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-BCMA antibody-encoding vectors. Saccharomyces cerevisiae, or common baker's yeast, is a commonly used lower eukaryotic host microorganism. However, a number of other genera, species, and strains are available and useful, such as Spodoptera frugiperda (e.g., SF9), Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-BCMA antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low. The antibodies produced in a cell-free system may be aglycosylated depending on the source of the cells.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO 1, 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 11. Pharmaceutical Compositions and Methods of Administration

The antibody conjugates provided herein can be formulated into pharmaceutical compositions using methods available in the art and those disclosed herein. Any of the antibody conjugates provided herein can be provided in the appropriate pharmaceutical composition and be administered by a suitable route of administration.

The methods provided herein encompass administering pharmaceutical compositions comprising at least one antibody conjugate provided herein and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions or antibody conjugates provided herein may be administered by any route known in the art. Exemplary routes of administration include, but are not limited to, the inhalation, intraarterial, intradermal, intramuscular, intraperitoneal, intravenous, nasal, parenteral, pulmonary, and subcutaneous routes. In some embodiments, a pharmaceutical composition or antibody conjugate provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In some embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibody conjugates.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific antibody in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody conjugate, since, in some embodiments, water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an antibody or antibody-conjugate will decompose. Such excipients, which are referred to herein as “stabilizers,” include, but are not limited to, antioxidants such as ascorbic acid, pH buffers, or salt buffers.

11.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

11.2. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amount of the antibody conjugate or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the antibody conjugate provided herein, based on weight of the antibody, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or for times weekly. It may be necessary to use dosages of the antibody conjugate outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody conjugate or composition provided herein followed by one or more maintenance doses.

In certain embodiments, a dose of an antibody conjugate or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

11.3. Combination Therapies and Formulations

In certain embodiments, provided are compositions, therapeutic formulations, and methods of treatment or uses comprising any of the antibody conjugates provided herein in combination with one or more chemotherapeutic agents disclosed herein, and methods of treatment comprising administering such combinations to subjects in need thereof. Examples of chemotherapeutic agents include, but are not limited to, Bendamustine (TREANDA®, Cephalon), Venetoclax (VENCLEXTA®, Abbvie, Genentech), Denosumab (XGEVA®, Amgen; PROLIA®, Amgen), Carfilzomib (KYPROLIS®, Amgen), Ixazomib (NINLARO®, Takeda), Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially uncialamycin, calicheamicin gammall, and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pladienolide B, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

In certain embodiments, provided are compositions, therapeutic formulations, and methods of treatment or uses comprising any of the antibody conjugates provided herein in combination with one or more PD-1 or PD-L1 inhibitors, and methods of treatment comprising administering such combinations to subjects in need thereof. In some embodiments, the one or more PD-1 or PD-L1 inhibitors comprise a small molecule blocker of the PD-1 or PD-L1 pathway. In some embodiments, the one or more PD-1 or PD-L1 inhibitors comprise an antibody that inhibits PD-1 or PD-L1 activity. In some embodiments, the one or more PD-1 or PD-L1 inhibitors are selected from the group consisting of: CA-170, BMS-8, BMS-202, BMS-936558, CK-301, and AUNP12. In some embodiments, the one or more PD-1 or PD-L1 inhibitors are selected from the group consisting of: avelumab, nivolumab, pembrolizumab, atezolizumab, durvalumab, AMP-224 (GlaxoSmithKline), MEDI0680/AMP-514 (AstraZeneca), PDR001 (Novartis), cemiplimab, TSR-042 (Tesaro, GlaxoSmithKline), Tizlelizumab/BGB-A317 (Beigene), CK-301 (Checkpoint Therapeutics), BMS-936559 (Bristol-Meyers Squibb), cemiplimab (Regeneron), camrelizumab, sintilimab, toripalimab, genolimzumab, and A167 (Sichuan Kelun-Biotech Biopharmaceutical). In some embodiments, the one or more PD-1 or PD-L1 inhibitors are selected from the group consisting of: MGA012 (Incyte/MacroGenics), PF-06801591 (Pfizer/Merck KGaA), LY3300054 (Eli Lilly), FAZ053 (Novartis), PD-11 (Novartis), CX-072 (CytomX), BGB-A333 (Beigene), BI 754091 (Boehringer Ingelheim), JNJ-63723283 (Johnson and Johnson/Jannsen), AGEN2034 (Agenus), CA-327 (Curis), CX-188 (CytomX), STI-A1110 (Servier), JTX-4014 (Jounce), AM0001 (Armo Biosciences, Eli Lilly), CBT-502 (CBT Pharmaceuticals), FS118 (F-Star/Merck KGaA), XmAb20717 (Xencor), XmAb23104 (Xencor), AB122 (Arcus Biosciences), KY1003 (Kymab), RXI-762 (RXi). In some embodiments, the one or more PD-1 or PD-L1 inhibitors are selected from the group consisting of: PRS-332 (Pieris Pharmaceuticals), ALPN-202 (Alpine Immune Science), TSR-075 (Tesaro/Anaptys Bio), MCLA-145 (Merus), MGD013 (Macrogenics), MGD019 (Macrogenics), R07121661 (Hoffman-La Roche), LY3415244 (Eli Lilly). In some embodiments, the one or more PD-1 or PD-L1 inhibitors are selected from an anti-PD1 mono-specific or bi-specific antibody described in, for example, WO 2016/077397, WO 2018/156777, and International Application No. PCT/US2013/034213, filed May 23, 2018.

In certain embodiments, provided are compositions, therapeutic formulations, and methods of treatment or uses comprising any of the antibody conjugates provided herein in combination with one or more LAG3 inhibitors, and methods of treatment comprising administering such combinations to subjects in need thereof. In some embodiments, the one or more LAG3 inhibitors comprise a small molecule blocker of the LAG3 pathway. In some embodiments, the one or more LAG3 inhibitors comprise an antibody that inhibits LAG3 activity. In some embodiments, the one or more LAG3 inhibitors are selected from the group consisting of: IMP321 (Eftilagimod alpha, Immutep), relatilimab (Brisol-Myers Squibb), LAG525 (Novartis), MK4280 (Merck), BI 754111 (Boehringer Ingelheim), REGN3767 (Regeneron/Sanofi), Sym022 (Symphogen) and TSR-033 (Tesaro/GSK).

In certain embodiments, provided are compositions, therapeutic formulations, and methods of treatment or uses comprising any of the antibody conjugates provided herein in combination with one or more TIM3 inhibitors, and methods of treatment comprising administering such combinations to subjects in need thereof. In some embodiments, the one or more TIM3 inhibitors comprise a small molecule blocker of the TIM3 pathway. In some embodiments, the one or more TIM3 inhibitors comprise an antibody that inhibits TIM3 activity. In some embodiments, the one or more TIM3 inhibitors are selected from the group consisting of: TSR-022 (Tesaro), LY3321367 (Eli Lilly), Sym023 (Symphogen) and MBG453 (Novartis).

In certain embodiments, provided are compositions, therapeutic formulations, and methods of treatment or uses comprising any of the antibody conjugates provided herein in combination with one or more CD73 inhibitors, and methods of treatment comprising administering such combinations to subjects in need thereof. In some embodiments, the one or more CD73 inhibitors comprise a small molecule blocker of the CD73 pathway. In some embodiments, the one or more CD73 inhibitors comprise an antibody that inhibits CD73 activity. In some embodiments, the one or more CD73 inhibitors are selected from the group consisting of: MEDI9447 (Medimmune), AB680 (Arcus), and BMS-986179 (Bristol-Myers Squibb).

In certain embodiments, provided are compositions, therapeutic formulations, and methods of treatment or uses comprising any of the antibody conjugates provided herein in combination with one or more CD39 inhibitors, and methods of treatment comprising administering such combinations to subjects in need thereof. In some embodiments, the one or more CD39 inhibitors comprise a small molecule blocker of the CD39 pathway. In some embodiments, the one or more CD39 inhibitors comprise an antibody that inhibits CD39 activity. In some embodiments, the one or more CD39 inhibitors are selected from the group consisting of: CPI-444 (Corvus), PBF-509 (Pablobio, Novartis), MK-3814 (Merck), and AZD4635 (AstraZeneca).

In certain embodiments, the antibody conjugates provided herein are administered in combination with VELCADE® (bortezomib), KYPROLIS® (Carfilzomib), NINLARO® (Ixazomib). In certain embodiments, the antibody conjugates provided herein are administered in combination with FARYDAK® (panobinostat). In certain embodiments, the antibody conjugates provided herein are administered in combination with DARZALEX® (daratumumab). In certain embodiments, the antibody conjugates provided herein are administered in combination with EMPLICITI® (elotuzumab). In certain embodiments, the antibody conjugates provided herein are administered in combination with AREDIA® (pamidronate) or ZOMETA® (zolendronic acid). In certain embodiments, the antibody conjugates provided herein are administered in combination with XGEVA® (denosumab) or PROLIA® (denosumab).

In certain embodiments, the antibody conjugates provided herein are administered in combination with a gamma secretase inhibitor (GSI), e.g., avagacestat (BMS-708163; Bristol-Myers Squib), MK-0752 (Merck & Co.), R04929097 (Roche), semagacestat (LY-450139; Eli Lilly & Co.), DAPT (N—[N-(3,5-Difluorophenylacetyl-L-alanyl)]-S-phenylglycine t-Butyl ester), L685,458, compound E ((s, s)-2-(3,5-Difluorophenyl)-acetylaminol-N-(1-methyl-2-oxo-5-phenyl-2,3-dihydro-1H-benzo[e][1,4]diazepin-3-yl)-propionamide), DBZ (dibenzazepine), JLK6 (7-amino-4-chloro-3-methoxyisocoumarin), or [11-endo]-N-(5,6,7,8,9,10-hexahydro-6,9-methano benzo[9][8]annulen-11-yl)-thiophene-2-sulfonamide.

The agents administered in combination with the antibody conjugates disclosed herein can be administered just prior to, concurrent with, or shortly after the administration of the antibody conjugates. In certain embodiments, the antibody conjugates provided herein are administered on a first dosing schedule, and the one or more second agents are administered on their own dosing schedules. For purposes of the present disclosure, such administration regimens are considered the administration of an antibody conjugate “in combination with” an additional therapeutically active component. Embodiments include pharmaceutical compositions in which an antibody conjugate disclosed herein is co-formulated with one or more of the chemotherapeutic agents, PD-1 inhibitors, or PD-L1 inhibitors disclosed herein.

### 12. Therapeutic Applications

For therapeutic applications, the antibody conjugates of the invention are administered to a mammal, generally a human, in a pharmaceutically acceptable dosage form such as those known in the art and those discussed above. For example, the antibody conjugates of the invention may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibody conjugates also are suitably administered by peritumoral, intralesional, or perilesional routes, to exert local as well as systemic therapeutic effects. The intraperitoneal route may be particularly useful, for example, in the treatment of ovarian tumors.

The antibody conjugates provided herein may be useful for the treatment of any disease or condition involving BCMA. In some embodiments, the disease or condition is a disease or condition that can be diagnosed by overexpression of BCMA. In some embodiments, the disease or condition is a disease or condition that can benefit from treatment with an anti-BCMA antibody. In some embodiments, the disease or condition is a cancer. In some embodiments, the disease or condition is a leukemia, a lymphoma, or multiple myeloma.

Any suitable cancer may be treated with the antibody conjugates provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some embodiments, the disease to be treated with the antibody conjugates provided herein is gastric cancer, colorectal cancer, renal cell carcinoma, cervical cancer, non-small cell lung carcinoma, ovarian cancer, uterine cancer, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In some embodiments, the disease is ovarian cancer. In some embodiments, the disease is breast cancer. In some embodiments, the disease is lung cancer. In some embodiments, the disease is head and neck cancer. In some embodiments, the disease is renal cell carcinoma. In some embodiments, the disease is brain carcinoma. In some embodiments, the disease is endometrial carcinoma.

In certain embodiments, the disease to be treated with the antibody conjugates provided herein is multiple myeloma. In specific embodiments, the multiple myeloma is Stage I, Stage II, or Stage III according to the International Staging System or the Revised International Staging System. In certain embodiments, said multiple myeloma is newly-diagnosed multiple myeloma. In other embodiments, said multiple myeloma is relapsed or refractory multiple myeloma.

Under the International Staging System (ISS), the stages of multiple myeloma are as follows: Stage I: Serum beta-2 microglobulin<3.5 mg/L and serum albumin≥3.5 g/dL; Stage II: Not stage I or stage III; Stage III: Serum beta-2 microglobulin≥5.5 mg/L. Under the Revised International Staging System (R-ISS), the stages of multiple myeloma are as follows: Stage I: ISS stage I and standard-risk chromosomal abnormalities by fluorescence in situ hybridization (FISH)(that is, no high-risk) and serum lactate dehydrogenase (LDH) level at or below the upper limit of normal; Stage II: Not R-ISS stage I or III; Stage III: ISS stage III and either high-risk chromosomal abnormalities by FISH (for example, presence of del(17p) and/or translocation t(4;14) and/or translocation t(14;16)) or serum LDH level above the upper limit of normal.

Multiple myeloma may also be staged using the Durie-Salmon system. Under this system, multiple myeloma is classified as stage I, II, or III (1, 2, or 3). Each stage is further classified into A or B, depending on whether kidney function has been affected, with the B classification indicating significant kidney damage. Stage I: Patients show no symptoms; however, if the cancer has affected kidney function, the prognosis may be worse regardless of the stage. Factors characteristic of stage I include: Number of red blood cells is within or slightly below normal range; normal amount of calcium in the blood; low levels of M protein in the blood or urine; M protein<5 g/dL for IgG; <3 g/dL for IgA; <4 g/24 h for urinary light chain; and/or no bone damage on x-rays or only 1 bone lesion is visible. Stage II: More cancer cells are present in the body in stage II, and if kidney function is affected, then the prognosis worsens regardless of the stage. Criteria for stage II are defined as those that fit neither stage I nor stage III. Stage III: Many cancer cells are present in the body at stage III. Factors characteristic of this stage include: Anemia, with a hemoglobin<8.5 g/dL; hypercalcemia; advanced bone damage (3 or more bone lesions); high levels of M protein in the blood or urine; and/or M protein>7 g/dL for IgG; >5 g/dL for IgA; >12 g/24 h for urinary light chain.

### 13. Diagnostic Applications

In some embodiments, the antibody conjugates provided herein are used in diagnostic applications. For example, an anti-BCMA antibody conjugate may be useful in assays for BCMA protein. In some aspects the antibody conjugate can be used to detect the expression of BCMA in various cells and tissues. These assays may be useful, for example, in making a diagnosis and/or prognosis for a disease, such as a cancer.

In some diagnostic and prognostic applications, the antibody conjugate may be labeled with a detectable moiety. Suitable detectable moieties include, but are not limited to radioisotopes, fluorescent labels, and enzyme-substrate labels. In another embodiment, the anti-BCMA antibody conjugate need not be labeled, and the presence of the antibody conjugate can be detected using a labeled antibody which specifically binds to the anti-BCMA antibody conjugate.

### 14. Affinity Purification Reagents

The antibody conjugates provided herein may be used as affinity purification agents. In this process, the antibody conjugates may be immobilized on a solid phase such a resin or filter paper, using methods well known in the art. The immobilized antibody conjugate is contacted with a sample containing the BCMA protein (or fragment thereof) to be purified, and thereafter the support is washed with a suitable solvent that will remove substantially all the material in the sample except the BCMA protein, which is bound to the immobilized antibody. Finally, the support is washed with another suitable solvent, such as glycine buffer, pH 5.0 that will release the BCMA protein from the antibody.

### 15. Kits

In some embodiments, an anti-BCMA antibody conjugate provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the anti-BCMA antibody conjugate. In some embodiments, the anti-BCMA antibody conjugate is provided in the form of a pharmaceutical composition.

### EXAMPLES

### Example 1

### Generation of Anti-BCMA Antibodies

### Generation and Phage Display Selection

Phage display was used to discover initial human antibody leads 2190-B01 and 2213-A06. Antibody Fab libraries were constructed using an optimized trastuzumab Fab sequence codon optimized in a modified, commercially available p3 phagemid vector (Antibody Design Labs). Briefly, the phagemid vector was modified to express Fab heavy chains as C-terminal p3 fusion proteins, and regulatory regions (start codons, restriction enzyme sites, periplasmic leader sequences) were optimized for Fab display levels. Libraries were constructed using a standard overlap extension PCR protocol with mutagenic primers targeting heavy chain complementary determining regions (CDRs). See Heckman and Pease, Nat. Protoc., 2007, 2:924-932. Libraries were rescued through electroporation in M13-K07 infected SS320 E. coli cells. Library selections were performed using standard phage display protocols. See Rajan & Sidhu, Methods Enzymol., 2012, 502:3-23; Marks & Bradbury, Methods Mol Biol., 2004, 248:161-76. Following multiple selection rounds, Fab heavy chain pools were transferred into cell-free expression vectors for expression as His6 and FLAG-tagged IgG1.

### Ribosome Display Selections

Ribosome display was used to discover initial human antibody leads 2137-A05 and 2137-C07. Ribosome display was also used to affinity mature 2137-C07, 2137-A05, 2190-B01, and 2213-A06 to generate improved derivative 2265, among others.

Antibody Fab libraries were constructed using a standard overlap extension PCR protocol with mutagenic primers targeting complementary determining regions (CDRs). See Heckman & Pease, supra. Selections for novel antibodies were performed using standard ribosome display protocols. See Hanes & Plückthun, Proc. Natl. Acad. Sci. U.S.A, 1997, 94:4937-4942. Specifically, Fab-based ribosome display selections were performed according to published protocols. See Stafford et al., 2014, Protein Eng. Des. Sel. 27:97-109; Dreier and Pluckthun, 2011, Methods Mol Biol 687:283-306. After multiple rounds of selection, the DNA from RT-PCR output was cloned into an optimized vector for cell-free expression using standard molecular biology techniques. See Yin et al., 2012, mAbs 4:217-225. All constructs were HIS- and FLAG-tagged to streamline purification and testing during screening.

Exemplary antibodies are reported in Table 6. Antibody 4 is also referred to as “Antibody 2265-F02” herein.

TABLE 6
Antibodies produced by ribosome and phage-display

| | | SEQ | | SEQ |
|---|---|---|---|---|
| Antibody | V_{H} | ID NO. | V_{L} | ID NO. |
| 4 | 2265-F02 | 13 | Trastuzumab | 14 |

### Example 2

### Primary Screening of Antibodies

### Primary ELISA Screening of Antibody Variants

Libraries of antibody variants generated by selection workflow were transformed into E. coli and grown on agar plates with antibiotic (Kanamycin). Individual colonies were grown in liquid broth (TB+antibiotic Kanamycin), and used as a template for DNA amplification via rolling circle amplification (RCA). The variants were then expressed in a cell-free protein synthesis reaction as described. See Yin et al., mAbs, 2012, 4:217-225. Briefly, cell-free extracts were treated with 50 μM iodoacetamide for 30 min at RT (20° C.) and added to a premix containing cell-free components (see Cai et al., Biotechnol Prg, 2015, 3:823-831), 10% (v/v) RCA DNA template (approximately 10 μg/mL DNA) for HC variants of interest, and 2.5 μg/mL of the trastuzumab LC. 60 μL cell free (CF) reactions were incubated at 30° C. for 12 hr on a shaker at 650 rpm in 96-well plates. 400-1500 colonies were screened, depending on the predicted diversity of different selection campaigns. Following synthesis, each reaction was diluted 1:200 and tested for binding to human or cynomolgus BCMA-Fc protein by ELISA. Briefly, BCMA-Fc (R&D Systems, Minneapolis, Minn.) was coated to 384-well Maxisorp plates in 0.1M bicarbonate (pH 8.9) and blocked with 1% BSA in PBST. Antibodies from a 1:200 diluted CF reaction were incubated on the plates, washed, and detected with HRP-conjugated anti-human Fab antibodies (Jackson ImmunoResearch, West Grove, Pa.) and Pierce Pico Supersignal ELISA substrate (ThermoFisher Scientific).

### High-Throughput Cell Binding

A high-throughput primary screen was performed to rapidly assess cell binding of antibodies produced in small-scale (60 μL) cell-free reactions. In this screen, four components were combined in equal volumes to a final volume of 100 μL/well in a U-bottom 96-well plate (Greiner Cat #650201) or flat bottom 384-well plate (Greiner Cat #781201). These components are: 1) BCMA-expressing NCI-H929 cells diluted in assay buffer (1×PBS+0.2% BSA, sterile filtered) to achieve a final concentration of 500,000 cells/well, 2) BCMA-negative MOLT-4 cells stained with CellTrace Oregon Green (Invitrogen Cat #34555) and diluted in assay buffer to achieve a final concentration of 500,000 cells/well, 3) a 1:50 dilution of cell-free reaction producing the antibody of interest diluted in assay buffer, and 4) a secondary anti-human antibody (AlexaFluor 647 AffiniPure F(ab′)2 Donkey anti-human IgG, Fc specific; Jackson ImmunoResearch Cat #709-606-098) diluted 1:100 in assay buffer. Plates were then incubated on ice for one hour. Cells were pelleted by spinning at 1500×g for 5 minutes and resuspended in assay buffer. High-throughput flow cytometry was then performed on resuspended cells on a FACS instrument (BD Biosciences FACSCanto II or BD Biosciences LSR II), and data was analyzed with FlowJo software. Antibody binding was assessed by the proportional level of secondary antibody signal (presumably due to binding to the antibody of interest) on NCIH929 BCMA-positive cells compared to the signal on MOLT-4 BCMA-negative cells.

### Example 3

### Secondary Screening of Antibodies

### Preparation of IgGs

The top leads from the initial round of screening were cultured and miniprepped via the Qiaprep 96 Turbo miniprep kit (Qiagen) according to manufacturer's instructions. 7.5 μg/mL miniprepped HC DNA and 2.5m/mL of the trastuzumab LC was added to 4 mL cell-free reactions and incubated overnight for 12 hr at 30° C., 650 rpm. Expressed variants from clarified cell-free reactions were purified via IMAC purification using a semi-automated high throughput batch purification method. Briefly, purifications were performed in a 96-well plate format where 50 μL/well of IMAC resin (Ni Sepharose High Performance, GE Healthcare) was equilibrated in IMAC binding buffer (50 mM Tris pH 8.0, 300 mM NaCl, 10 mM imidazole), incubated with 1 mL cell-free reaction for 15 minutes followed by two washes in IMAC binding buffer. His-tagged antibody variants were then eluted using 200 μL IMAC elution buffer (50 mM Tris pH 8.0, 300 mM NaCl, 500 mM imidazole) and buffer exchanged into PBS using a 96-well Zeba plate (7 kD MWCO, Thermofisher). Purified antibodies were quantified via high throughput capillary electrophoresis using the Labchip GXII (Perkin Elmer) against a Herceptin standard curve, according to manufacturer's instructions.

Preparation of scFvs

A single-chain antibody is made in either the V_{H}V_{L} or V_{L}V_{H} orientation with a linker sequence between the V_{H} and V_{L} domains. Typically scFv linkers are composed of (GGGGS)n (SEQ ID NO: 28) repeats where n=3, 4, 5, or 6 for linkers of 15, 20, 25, or 30 residues respectively. For cell-free expression, an N-terminal Met is added, but for mammalian expression a leader peptide is added. On the C-terminal end of the scFv, an Fc sequence can be added to extend in vivo half-life or the scFv can be used directly. An optional linker sequence can be incorporated between the scFv and the Fc. An exemplary scFv-Fc linker sequence is AAGSDQEPKSS (SEQ ID NO: 27). C-terminal affinity tags can optionally be added to facilitate purification and assay development. An exemplary affinity tag is a C-terminal FlagHis tag GSGDYKDDDDKGSGHHHHHH (SEQ ID NO: 25). A stop codon is typically inserted at the end of the sequence. An exemplary scFv can include an N-terminal Met residue, a V_{H} domain, a GGGGSGGGGSGGGGS (SEQ ID NO: 26) linker, a V_{L} domain, an AAGSDQEPKSS (SEQ ID NO: 27) linker, an Fc domain, a FlagHis tag, and a stop codon.

### Differential Scanning Fluorimetry

A protein thermal shift assay was carried out by mixing the protein to be assayed with an environmentally sensitive dye (SYPRO Orange, Life Technologies Cat #S-6650) in a phosphate buffered solution (PBS), and monitoring the fluorescence of the mixture in real time as it underwent controlled thermal denaturation. Protein solutions between 0.2-2 mg/mL were mixed at a 1:1 volumetric ratio with a 1:500 PBS-diluted solution of SYPRO Orange (SYPRO Orange stock dye is 5000× in DMSO). 10 μL aliquots of the protein-dye mixture were dispensed in quadruplicate in a 384-well microplate (Bio-Rad Cat #MSP-3852), and the plate was sealed with an optically clear sealing film (Bio-Rad Cat #MSB-1001) and placed in a 384-well plate real-time thermocycler (Bio-Rad CFX384 Real Time System). The protein-dye mixture was heated from 25° C. to 95° C., at increments of 0.1° C. per cycle (˜1.5° C. per minute), allowing 3 seconds of equilibration at each temperature before taking a fluorescence measurement. At the end of the experiment, the transition melting temperatures (TM1 and TM2) were determined using the Bio-Rad CFX manager software. TM1 represents the melting temperature of the Fc domain. TM2 represents the melting temperature of the Fab domain.

### Biacore Off-Rate and Kinetic Analysis

Anti-Fab or anti-Fc polyclonal antibodies were immobilized onto a CMS chip (GE Life Sciences) using amine coupling chemistry (from Amine Coupling Kit, GE Life Sciences). The immobilization steps were carried out at a flow rate of 25 μL/min in 1×HBS-EP+ buffer (GE Life Sciences; 10× Stock diluted before use). The sensor surfaces were activated for 7 min with a mixture of NHS (0.05 M) and EDC (0.2 M). The anti-Fab or anti-Fc antibodies were injected over all 4 flow cells at a concentration of 25 μg/ml in 10 mM sodium acetate, pH 4.5, for 7 min. Ethanolamine (1 M, pH 8.5) was injected for 7 min to block any remaining activated groups. An average of 12,000 response units (RU) of capture antibody was immobilized on each flow cell.

Off-rate and kinetic binding experiments were performed at 25° C. using 1×HBS-EP+ buffer. Test and control antibodies were injected over the anti-Fab or anti-Fc surface at concentrations of 5-10 μg/mL for 12 seconds at a flow rate of 10 μL/min on flow cells 2, 3 and 4, followed by a buffer wash for 30 seconds at the same flow rate. Kinetic characterization of antibody samples was carried out with a range of antigen concentrations from 1-100 nM and 1 injection of 0 nM antigen (for example, 100, 50, 25, 6.25, 1.56 and 0 nM). After capturing ligand (antibody) on the anti-Fab or anti-Fc surface, the analyte (human BCMA-Fc, cyno BCMA-Fc, or human BCMA from R&D Systems, custom protein production, or Sigma Aldrich, respectively) was bound for 180 seconds, followed by a 600 second dissociation phase at a flow rate of 50 μL/min. Between each ligand capture and analyte binding cycle, regeneration was carried out using 2 injections of 10 mM glycine pH 2.0 for 30 seconds at 30 μL/min, followed by a 30 second buffer wash step.

The data was fit with the Biacore T200 Evaluation software, using a 1-1 Langmuir binding model. K_{D} (affinity, nM) was determined as a ratio of the kinetic rate constants calculated from the fits of the association and dissociation phases.

### Cell Lines and Cell Culture Conditions

NCI-H929, U266B1, MOLT-4 and ARP-1, were obtained from ATCC and the Keats Lab (Tgen, Phoenix, Ariz.). 293T-cynoBCMA and 293T-ratBCMA recombinant cells were generated by transfecting 293T cells with a plasmid containing cynomolgus or rat BCMA cDNA sequences and selecting for the highest stable expression of cynomolgus BCMA or rat BCMA on the cell surface. NCI-H929, U266B1, and MOLT-4 cells were maintained in RPMI-1640 (Cellgro-Mediatech; Manassas, Va.) supplemented with 20% heat-inactivated fetal bovine serum (Hyclone; Thermo Scientific; Waltham, Mass.), 1% Penicillin/Streptomycin (Cellgro-Mediatech; Manassas, Va.), and 2 mmol/L-glutamax (Life Technology; Carlsbad, Calif.). 293T-cynoBCMA and 293T-ratBCMA cells were maintained in Ham's F-12-high glucose DMEM (50-50) (Cellgro-Mediatech; Manassas, Va.) supplemented with 10% heat-inactivated fetal bovine serum (Hyclone; Thermo Scientific; Waltham, Mass.), 1% Penicillin/Streptomycin (Cellgro-Mediatech; Manassas, Va.), and 2 mmol/L-glutamax (Life Technology; Carlsbad, Calif.).

### Cell Binding Experiments

Variants for which sufficient protein was purified in secondary screening were tested in a fluorescence-activated cell sorting (FACS) cell-binding assay. BCMA positive NCI-H929 and 293T-cynoBCMA cells and BCMA negative 293T cells were used to screen for FACS binders. 293T cells were treated with 1 μM DAPT 24 hours prior to cell binding to prevent BCMA shedding. 6-12 point dilutions of anti-BCMA variants starting from concentrations of about 100-200 nM antibody were dispensed into each well using a BioMekFX (Beckman Coulter). Cells were then incubated on ice for 1 hr, washed with FACS buffer and incubated for 1 hr on ice with 50 mL FACS buffer containing 2.5 μg/ml Alexa647-conjugated Goat Anti-Human IgG dispensed using BioMekFX (Beckman Coulter). Cells were then washed 2× with FACS buffer and fixed for 10 minutes in 200 ml PBS with 2% paraformaldehyde (PFA) prior to fluorescence detection. Samples were acquired using a Beckton Dickinson LSRII FACS. Geometric Mean Fluorescence Intensity of BCMA antibody binding was analyzed using FlowJo® software (Tree Star, Inc.).

### Cell-Killing Analysis

The internalization of the antibodies was evaluated by drugs conjugated to secondary antibodies in a cell killing assay on BCMA positive cells. BCMA-positive cell lines ARP-1 and U266B1 were used to screen for internalizing leads. Cells were washed twice with calcium and magnesium-free Dulbecco's phosphate-buffered saline (DPBS), harvested with Accutase® (Innovative Cell Technologies; San Diego, Calif.) and counted by the Vi-CELL Cell Viability Analyzers (Beckman Coulter, Brea, Calif.). A total of 12,500 cells in a volume of 25 microliter were seeded in a 384-well flat bottom white polystyrene plate (Greiner Bio-One, Monroe, N.C.) on the day of assay. Lead antibodies were formulated at 4×starting concentration in the cell culture medium and filtered through MultiScreenHTS 96-Well Filter Plates (Millipore; Billerica, Mass.). 12.5 μL of the serial diluted antibody (1:3 serial dilution starting from 100 nM) was added into treatment wells and 12.5 μL of an anti-human nanobody conjugated to according to Conjugate P (hemiasterlin via a cleavable linker) or according to Conjugate M (maytansinoid via a non-cleavable linker) was then added into each well at a fixed final concentration of 20 nM. Assay plates were cultured at 37° C. in a CO₂ incubator for 72 hrs before assay. For cell viability measurement, 30 μL of Cell Titer-Gb® reagent (Promega Corp. Madison, Wis.) was added into each well, and plates were processed as per product instructions. Relative luminescence was measured on an ENVISION® plate reader (Perkin-Elmer; Waltham, Mass.). Relative luminescence readings were converted to % viability using untreated cells as controls. Data was fitted with non-linear regression analysis, using a log(inhibitor) vs. response-variable slope, 4 parameter fit with GraphPad Prism (GraphPad v 5.0, Software; San Diego, Calif.). Data was expressed as relative cell viability (ATP content) % vs. dose of antibody.

### Example 4

### Characteristics of Illustrative Anti-Bcma Antibodies

Tables 7A and 7B show results obtained with antibodies produced by ribosome and phage-display of initial leads and after affinity maturation.

TABLE 7A
Antibodies from ribosome and phage-display.

| | | | | | ARP-1, | | U266B1, | |
|---|---|---|---|---|---|---|---|---|
| | NCI-H929 | | | | Conjugate | | Conjugate | |
| | (BCMA+ | | 293T- | | M 2° | | M 2° | |
| | cells) | | cynoBCMA | | antibody | | antibody | |
| | cell binding | | cell binding | | cell killing | | cell killing | |
| Fab-HC | Bₘₐₓ | Kd | Bₘₐₓ | Kd | EC₅₀ | Span | EC₅₀ | Span |
| Variant ID | (MFI) | (nM) | (MFI) | (nM) | (nM) | (%) | (nM) | (%) |
| 2265-F02 | 11728 | 5.2 | 23759 | 6.2 | 1.9 | 55 | 0.9 | 58 |

NK = no killing

TABLE 7B
Antibodies from ribosome and phage-display.

| | Thermostability | Biacore, human BCMA-Fc | | | Biacore, cyno BCMA-Fc | | |
|---|---|---|---|---|---|---|---|
| Fab-HC | Fab TM2 | kₐ | k_{d} | K_{D} | kₐ | k_{d} | K_{D} |
| Variant ID | (° C.) | (1/Ms) | (1/s) | (M) | (1/Ms) | (1/s) | (M) |
| 2265-F02 | 85.0 | 5.87E+05 | 2.93E−04 | 4.99E−10 | 3.15E+05 | 1.16E−03 | 3.68E−09 |

ND = not detected

### Example 5

### Antibody-Drug Conjugation and Dar Ratio Determination

Antibody-drug conjugation is described in Zimmerman E S, et al. 2014, Bioconjugate Chem., 25 (2), pp 351-361. Briefly, purified anti-BCMA antibody variants were conjugated to a cytotoxic agent. Stock drug was dissolved in DMSO to a final concentration of 5 mM. The compound was diluted with PBS to 1 mM and then added to the purified protein sample in to final drug concentration of 100 μM. Mixture was incubated at RT (20° C.) for 17 hours. Unincorporated drug was removed by passing the reaction sample through a 7000 MWCO resin in Zeba plates (Thermo Scientific) equilibrated in formulation buffer. Filtrate was then passed through a MUSTANG® Q plate (Pall Corp.) to remove endotoxin.

Following purification, the purified antibody or antibody drug conjugate samples were quantified on a Caliper GXII system by comparing with by mass standards of HERCEPTIN® run on the same Protein Express LabChip (Caliper Life Sciences #760499). Samples were prepared for analysis as specified in the Protein Express Reagent Kit (Caliper Life Sciences #760328) with the exception that the samples (mixed in sample buffer+50 mM NEM) were heated at 65° C. for 10 minutes prior to analysis on the Caliper system.

Antibody drug conjugates were reduced in with 10 mM TCEP (Pierce) for 10 min at 37° C. Add 30 uL of TA30 (30% Acetonitrile, 70% of 0.1% Trifluoroacetic acid) to the reduced sample. Dissolve 20 mg of super-DHB (Sigma, part No. 50862) into TA50 (50% acetonitrile, 50% of 0.1% trifluoroacetic acid) to generate a sample matrix. Next add 0.5 uL of sample in TA30 to 0.8 uL of super-DHB matrix in TA50 and deposit onto MALDI sample plate. Spectra were acquired on a Bruker Autoflex Speed MALDI instrument with the following initial settings: Mass range 7000-70000 Da, sample rate and digitizer settings of 0.05, 0.1, 0.5, 1, 2, with realtime smoothing set at High and no baseline offset adjustment. High voltage switched On and Ion source 1 adjusted to 20 kV. Pulse ion extraction at 200 ns, matrix suppression on deflection and suppress up to 6000 Da. Peak detection algorithm is centroid with signal to noise threshold at 20, peak width at 150m/z height at 80% with baseline subtraction TopHat. Smoothing algorithm is SavtzkyGolay with width of 10m/z and cycles of 10. The drug-antibody ratio (DAR) for all samples was determined as a weighted average of the deconvoluted mass spectrum area under the curve for each conjugate.

### Example 6

### In Vitro Plasma Stability

In this example, the in vitro stability of conjugate 4 was evaluated in plasma from human, cynomolgus monkey and mouse. The linker-warhead stability was measured by a LC/MS based-assay utilizing affinity-captured antibody. ADCs (50 μL at 100 μg/mL) were incubated with PBS or plasma (lithium-heparin) samples from human, cynomolgus monkey or mouse for different lengths of time (0, 2, 24, 72, 168, 336 and 504 hrs). The samples were taken out at predetermined time points and added to Streptavidin Mag Sepharose Beads (GE Healthcare, Cat #28-9857-99,) that have been coated with Biotin-(Fab)2 Goat Anti-Human IgG, Fcγ fragment specific (Jackson Immnoresearch, cat #109-066-098) antibodies (for PBS, cyno and mouse plasma samples) or Biotinylated human BCMA ECD (for human plasma samples) (10 ug/sample). The plasma sample/bead mixtures were incubated at room temperature for 2 hours with gentle rotation. The beads were then washed three times in 1 mL HBS-E buffer, followed by two washes with 1 mL water. Elution of the captured ADCs was performed with addition of 25 μL of 1% formic acid solution at room temperature for 5 min. The released antibody was removed from the beads and neutralized with 15 μL of 1M Tris-HCl (pH 9.0).

The DARs of the pull-down ADCs were acquired on an Agilent 6520A Accurate Mass Q-TOF MS connected to an Agilent 1200 series HPLC system with a Binary SL pump. Additional chromatographic traces were acquired on an Agilent DAD at 278 and 214 nm. The pull-down method loading was optimized to that the entire volume of sample (40 μL) was injected onto an Agilent Advance Bio Desalting HPLC cartridge (2.1×12.50 mm) at 80° C. and 0.4 mL/min. Standard mobile phases for LC-MS were employed: A: 0.1% formic acid in water; B: 0.1% formic acid in acetonitrile. After a 1 min desalting time at 10% B protein was eluted from the cartridge from 1.5-4.5 min from 65-80% B. Carry over was prevented by running a cleaning grading between each injection.

All spectra were extracted and combined from a single TIC peak using MassHunter Qualitative (B.06.00) from Agilent. The spectra were deconvoluted using the Maximum Entropy algorithm in MassHunter Qualitative and identity confirmed from the observed neutral mass. Deconvolution was restricted to the ions originating from the fully assembled antibody, a mass range of 140,000-160,000 Da was searched with a mass step of 1.0 Da.

Peak areas were assigned in DAR Calculator B.1.0 (Agilent Technologies). Where automatic peak picking failed, peaks were defined manually. The resulting peak table was exported to an Excel worksheet and the DAR values reassigned as appropriate. In cases where drug-linker degradation was observed, only the remaining drugs on the product species were counted as active. For example, an antibody with one full drug-linker and just a linker (degraded from a 2-drug species) was considered equivalent to a one-drug species. The overall DAR value was calculated as a weighted average of deconvoluted peak areas. Overall DAR values for replicate samples were averaged together.

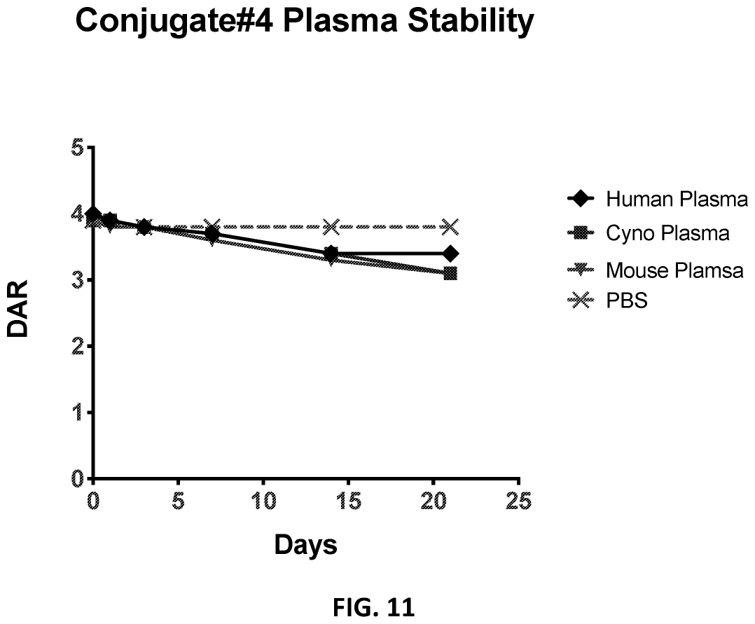

Exemplary plasma stability results are provided for Conjugate 4 in FIG. 11.

### Example 7

### Evaluation of Dose Response Relationship of BCMA ADC Variants in ARP-1 Multiple Myeloma Tumors

A study was conducted to compare the efficacy of Conjugate 4 (described in Table 8) in subcutaneous ARP-1 multiple myeloma tumors.

TABLE 8
List of test articles

| Conjugate | Description |
|---|---|
| 4 | Antibody 2265-F02 conjugated to |
| | a non-cleavable maytansine warhead |
| | (Conjugate M) at Y180/F404 site (DAR4) |

Anti-BCMA ADCs were generated by conjugating linker payload to para-Azido-Methyl-Phenylalanine (pAMF) at the F404 site of antibodies described herein. Conjugate 1, a surrogate ADC for GSK2857916 (GSK, Trudel et al., 2018, Lancet Oncol. 19:1641-1653; Trudel et al., 2019, Blood Cancer Journal 9:37), was generated by conjugating a maleimido-caproyl monomethyl autistatin F (mc-MMAF) linker-warhead to the anti-BCMA antibody J6M0. The J6M0 antibody was made with a CHO cell line, CHOEBNALT (Icosagen), and purified by ProA. The mc-MMAF linker-warhead and conjugated to J6M0 to produce Conjugate 1. Unlike GSK2857916, Conjugate 1 does not use an afucosylated antibody, which might enhance Fc-gammaRIII interactions.

Female severe combined immune deficient (SCID) Beige mice 9 weeks of age were anesthetized with isoflurane and implanted subcutaneously into the right hind flank with a 1:1 mixture of 1×10⁷ human ARP-1 MM cells and matrigel. Randomization and start of treatment was initiated when the average tumor size was approximately 150 mm³ (corresponding to 15 days post-implantation). The treatment groups are outlined in Table 9. All test articles were formulated in 10 mM citrate pH 6.0, 10% sucrose. Body weight and tumor size were monitored 1-2× per week. Primary study endpoint was when the mean tumor size of the vehicle control group was >1,500 mm³.

TABLE 9
List of Treatment Groups

| | | Dose | Dosing | | |
|---|---|---|---|---|---|
| Group | Treatment | (mg/kg) | frequency | Route | N |
| 1 | PBS | — | single | IV | 8 |
| 2 | Conjugate 4 | 0.1 | single | IV | 8 |
| 3 | Conjugate 4 | 0.5 | single | IV | 8 |
| 4 | Conjugate 4 | 2 | single | IV | 8 |
| 5 | Conjugate 4 | 8 | single | IV | 8 |
| 10 | Conjugate 1 | 2 | single | IV | 8 |

Body weight and tumor size were analyzed using a one-way analysis of variance (ANOVA) with Dunnett's multiple comparison test. A probability of less than 5% (p<0.05) was considered statistically significant.

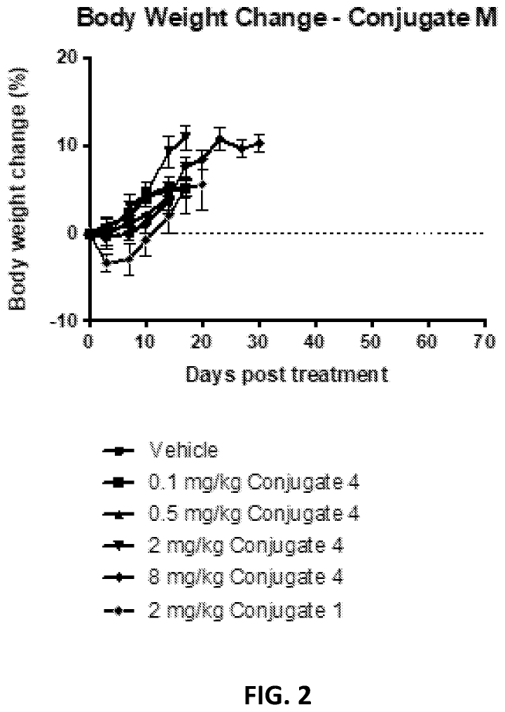

In this study, animals bearing established ARP-1 tumors were treated once with 4 dose levels of Conjugate 4 ranging from 0.1 to 8 mg/kg or 2 mg/kg Conjugate 1. All test articles were well tolerated and none exhibited any toxicity based on body weight loss FIG. 2.

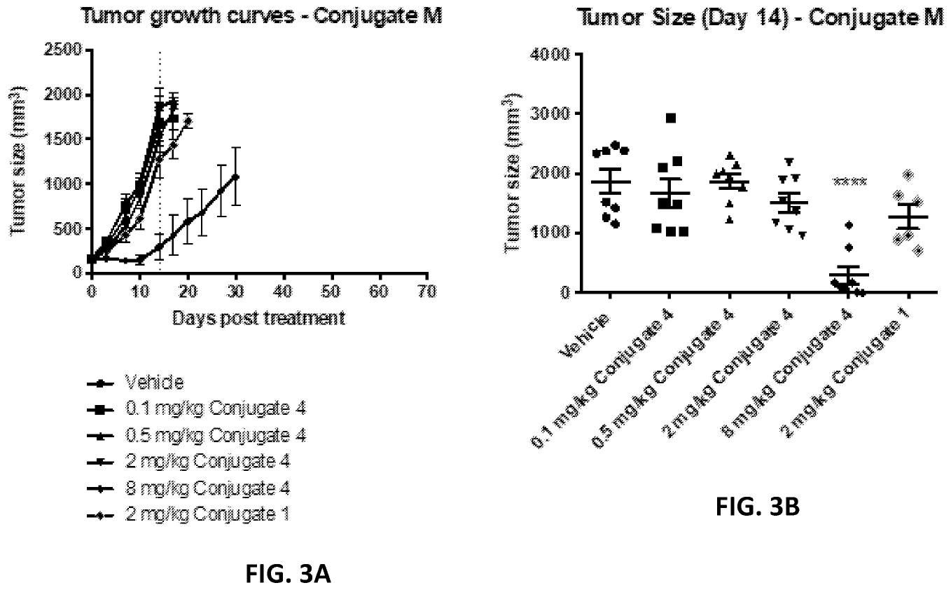

The effects of treatment on ARP-1 tumor growth are illustrated in FIG. 3A and FIG. 3B and show a positive correlation between increasing activity and dose for both drugs. Both BCMA ADC variants had little to no activity, similar to vehicle control, at the two lower doses (0.1 and 0.5 mg), while moderate activity was observed with 2 mg/kg (FIG. 3A). The highest Conjugate 4 dose at 8 mg/kg resulted in tumor stasis with tumor regrowth observed approximately 10 days after treatment (FIG. 3A).

Results from this study show that activity of Conjugate 4 was not statistically different compared to Conjugate 1 in this model.

### Example 8

### Evaluating the Dose Response Relationship of BCMA ADC Variants Conjugates 4 and 5 in the Disseminated MM.1S Multiple Myeloma Model

A study was conducted to evaluate the efficacy of Conjugate 4 in the disseminated MM.1S model in NSG mice.

Female NOD severe combined immune deficient (SCID) gamma (NSG) mice 8-9 weeks of age were inoculated with 5×10⁶ multiple myeloma MM.1S cells into the tail vein. Randomization by body weight and start of treatment was initiated 7 days post tumor inoculation. The treatment groups are outlined in Table 10. All investigational test articles were formulated in 10 mM citrate pH 6.0, 10% sucrose. Groups 1-10 (n=6/group) were monitored for survival endpoint characterized by >20% body weight loss and clinical signs including lethargy, hind limb paralysis or moribundity. Groups 11-20 (n=3/group) were used for bone marrow harvest and analysis of tumor burden on day 28 post tumor cell inoculation. For all groups, body weights were monitored 1-2×/week.

TABLE 10
List of Treatment Groups

| | | Dose | Dosing | | |
|---|---|---|---|---|---|
| Group | Treatment | (mg/kg) | frequency | Route | N |
| 1 | PBS | — | single | IV | 6 |
| 2 | Conjugate 4 | 0.02 | single | IV | 6 |
| 3 | Conjugate 4 | 0.1 | single | IV | 6 |
| 4 | Conjugate 4 | 0.5 | single | IV | 6 |
| 5 | Conjugate 4 | 2.5 | single | IV | 6 |
| 10 | Conjugate 1 | 0.5 | single | IV | 6 |
| 11 | PBS | — | single | IV | 3 |
| 12 | Conjugate 4 | 0.02 | single | IV | 3 |
| 13 | Conjugate 4 | 0.1 | single | IV | 3 |
| 14 | Conjugate 4 | 0.5 | single | IV | 3 |
| 15 | Conjugate 4 | 2.5 | single | IV | 3 |
| 20 | Conjugate 1 | 0.5 | single | IV | 3 |

Tumor burden was assessed and quantified by detection of hCD138 positive (hCD138+) cells in the bone marrow. Bone marrow cells from mouse femur and tibia were pooled and assessed for human CD138+ expression using the Alexa Fluor 647 mouse anti-human CD138 clone MI15 (BD Biosciences #562097) according to the manufacturer's protocol. CD138 is a specific surface antigen for MM and plasma cells in the bone marrow (Chilosi Met. Al. Mod. Pathol. Off. J. U. S. Can. Acad. Pathol. Inc (1999): 12, 1101-1106). Direct immunofluorescence flow cytometric analysis was performed using an LSRII flow cytometer and FACS Diva Software. Data was analyzed using Flowjo (Tree Star, Inc., Ashland, Oreg.).

Mean survival, survival delay, and tumor burden, during and at the study endpoint, were analyzed using a one-way analysis of variance (ANOVA) with Dunnett's multiple comparison test. A probability of less than 5% (p<0.05) was considered statistically significant.

In this study, animals bearing established MM.1S tumors were treated once with 4 dose levels of Conjugate 4 ranging from 0.02 to 2.5 mg/kg, or 0.5 mg/kg of surrogate Conjugate 1 on day 7 post inoculation.

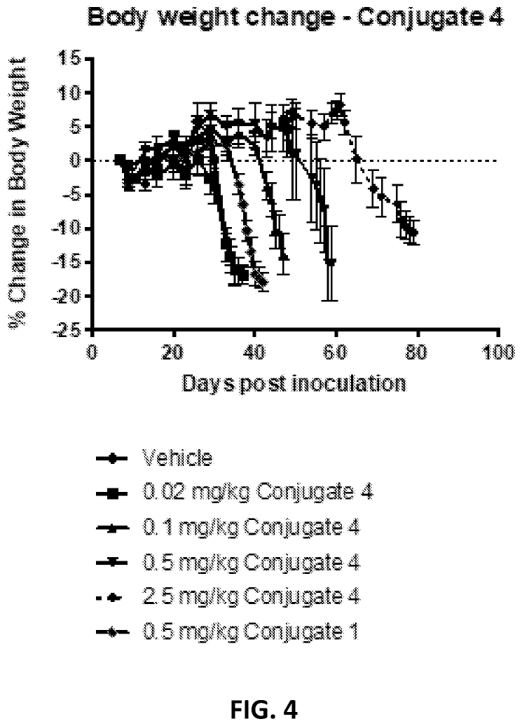

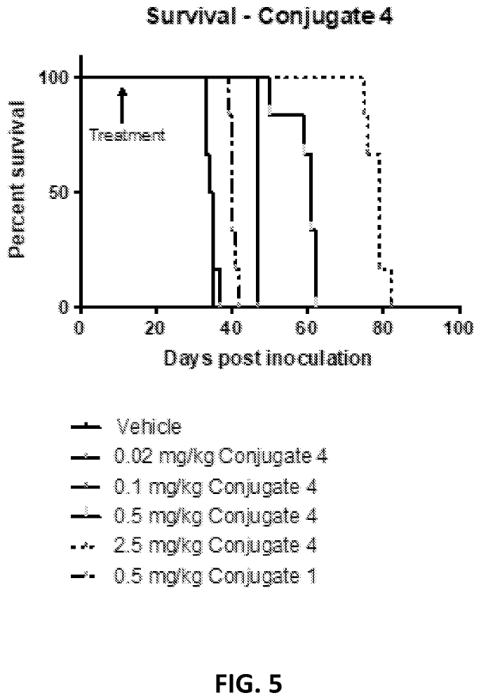

FIG. 4 shows all treatment groups induced minimal body weight loss (˜5% body weight loss) and were well tolerated. Body weight loss in vehicle control animals started on day 30, followed by progressive body weight loss (until >20%) coincident with development of clinical signs including hind-limb paralysis, piloerection, and lethargy. Survival curves are illustrated in FIG. 5. The mean survival for the vehicle group was 34.2 days. A linear increase in mean survival was observed with increasing Conjugate 4 doses starting at approximately day 43 with 0.1 mg/kg and up to approximately 77 days with 2.5 mg/kg (FIG. 5). All doses ≥0.1 mg/kg significantly increased survival compared to vehicle control (FIG. 3).

Results from this study show that Conjugate 4 in the disseminated MM.1S model was significantly more efficacious than an equivalent dose of Conjugate 1 in reducing tumor burden and prolonging survival.

### Example 9

### Evaluating the Efficacy of Conjugate 4 in Combination with Mm SOC Velcade/Bortezomib or Darzalex/Daratumumab in the Disseminated MM.15 Model in NSG Mice

A study was conducted to evaluate the efficacy of Conjugate 4 in combination with MM standard of care (SOC) agents Velcade and Daratumumab in the disseminated MM.15 model in NSG mice.

Female NOD severe combined immune deficient (SCID) gamma (NSG) mice 9-12 weeks of age were inoculated with 5×10⁶ multiple myeloma MM.1S cells into the tail vein. Randomization by body weight and start of treatment was initiated 7 days post tumor inoculation. The treatment groups are outlined in Table 11. All Sutro investigational test articles were formulated in 10 mM citrate pH 6.0, 10% sucrose. Clinical grade Daratumumab and Velcade (Pharmaceutical Buyers International) were formulated as per manufacturer's recommendations. Test articles were administered by intraperitoneal (IP) or intravenous (IV) injection. Body weights were monitored 1-2×/week. Study endpoint was survival and characterized by >20% body weight loss and clinical signs including lethargy, hind limb paralysis or moribundity.

TABLE 11
List of Treatment Groups

| | | Dose | Dosing | | |
|---|---|---|---|---|---|
| Group | Treatment | (mg/kg) | frequency | Route | N |
| 1 | Vehicle/PBS | NA | single | IV | 5 |
| 2 | Conjugate 4 | 0.25 | single | IV | 5 |
| 3 | Daratumumab | 3 | single | IP | 5 |
| 4 | Daratumumab | 10 | single | IP | 5 |
| 5 | Velcade | 0.8 | q7dx2 | IP | 5 |
| 6 | 0.25 mg/kg Conjugate | See single agent | | | 5 |
| | 4 + 3 mg/kg Daratumumab | treatments | | | |
| 7 | 0.25 mg/kg Conjugate | See single agent | | | 5 |
| | 4 + 10 mg/kg Daratumumab | treatments | | | |
| 8 | 0.25 mg/kg Conjugate | See single agent | | | 5 |
| | 4 + 0.8 mg/kg Velcade | treatments | | | |
| 9 | Conjugate 4 | 10 | single | IV | 5 |

Mean survival (days) was analyzed to compare the effect of treatment versus vehicle or relevant treatment groups to each other using one-way analysis of variance (ANOVA) with the Dunnett's and Sidak's multiple comparison tests, respectively. A probability of less than 5% (p<0.05) was considered as significant.

In this study, animals bearing established MM.1S tumors were treated on day 7 post-inoculation with 0.25 mg/kg Conjugate 4 (single dose), 3 mg/kg Daratumumab (single dose), 10 mg/kg Daratumumab (single dose), 0.8 mg/kg Velcade (q7dx2), or a combination of 0.25 mg/kg Conjugate 4 with each dose of Daratumumab or Velcade. In addition, a single high dose of Conjugate 4 at 10 mg/kg was administered.

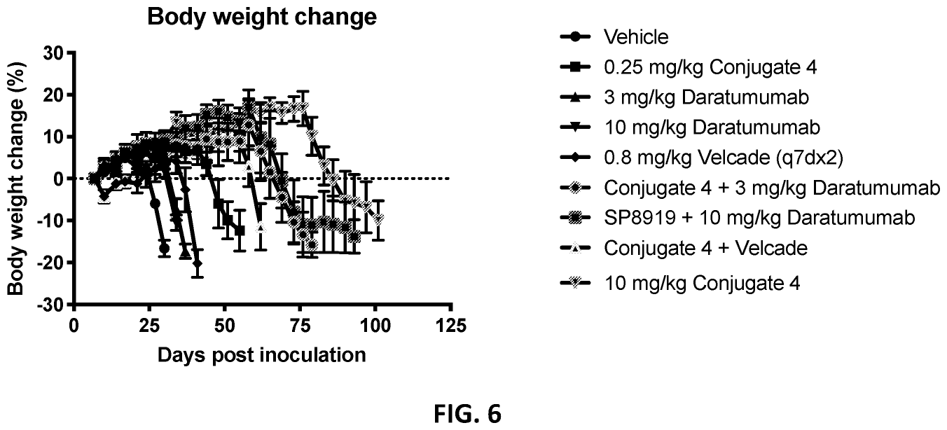

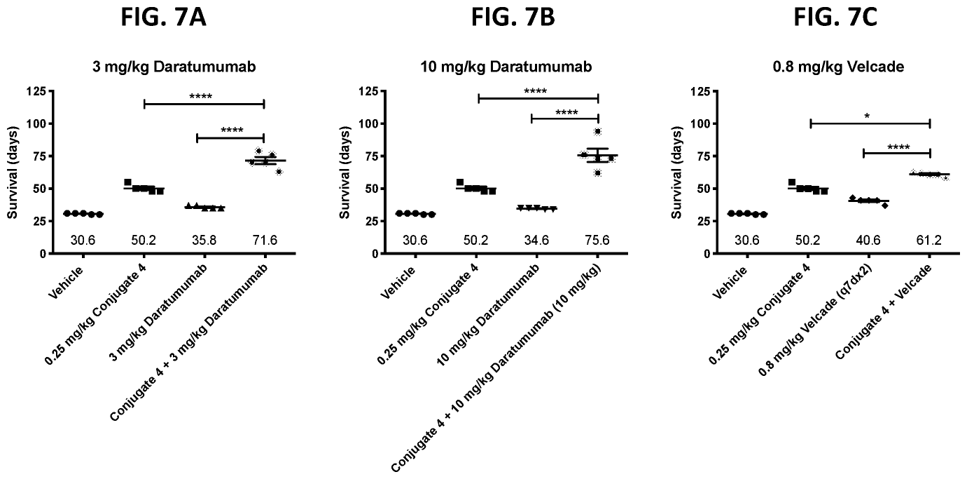

FIG. 6 shows all treatments initially induced minimal body weight loss (˜5% body weight loss) and were well tolerated. As expected in this model, body weight loss in vehicle control animals started on approximately day 24, followed by progressive body weight loss (until >20%) coincident with development of clinical signs including hind-limb paralysis, piloerection, and lethargy. FIG. 7A-7C shows Kaplan-Meier survival curves in response to 0.25 mg/kg Conjugate 4 and MM SOC therapeutics as single agents or combinations. The mean survival for the vehicle group was 30.6 days (FIG. 7A-7C). Single agent treatment with 0.25 mg/kg Conjugate 4 or 0.8 mg/kg Velcade resulted in significantly longer mean survival (50.2 and 40.6 days, respectively) compared to vehicle control (FIG. 7A). Co-administration of Conjugate 4+Velcade appeared to have an additive effect on mean survival at 61.2 days which was significantly different compared to either single agent. Meanwhile, single agent Daratumumab at 3 or 10 mg/kg had no significant effect on survival compared to vehicle control (FIG. 7A, FIG. 7B and FIG. 7C). However, Conjugate 4+Daratumumab at either dose resulted in significantly prolonged mean survival (71.6 and 75.6 days, respectively) compared to single agents alone (FIG. 7B and FIG. 7C). The lack of single agent Daratumumab efficacy suggests a synergistic effect in combination with Conjugate 4.

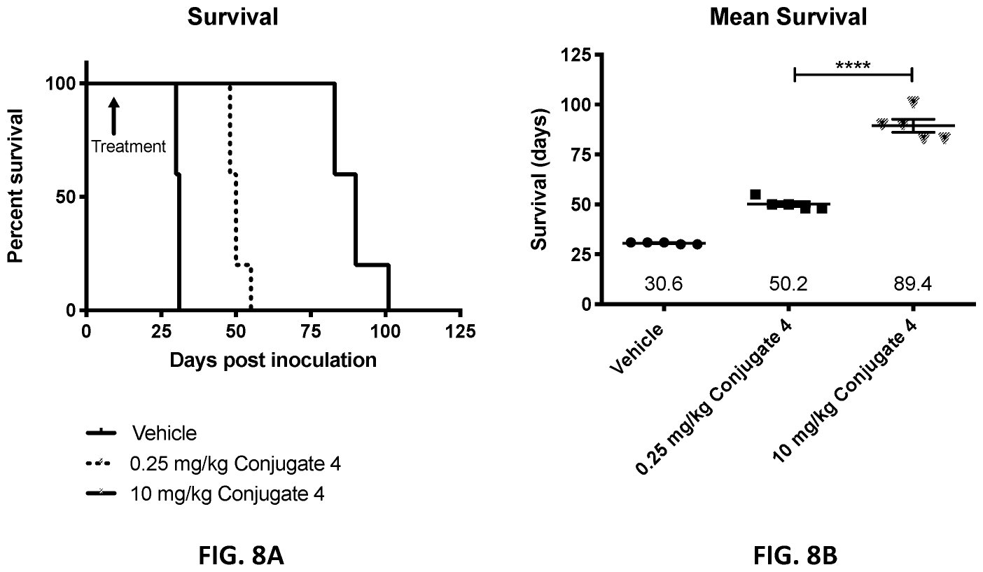

FIG. 8A shows Kaplan-Meier survival curves in response to a higher dose of Conjugate 4 at 10 mg/kg. Mean survival of animals treated with 10 mg/kg Conjugate 4 was 89.4 days, which was extended significantly compared to vehicle control or 0.25 mg/kg Conjugate 4 (FIG. 8B).

Results from this study show that Conjugate 4 in combination with Velcade or Daratumumab significantly potentiated efficacy compared to Conjugate 4 or MM SOC single agents alone. It should be noted that since NSG mice lack NK cells, the combination benefit observed with Daratumumab in this model may be attributed to its NK-independent functions (Phipps C et al., 2015, Ther. Adv. Hem. 63:120-127). In addition, treatment with 10 mg/kg Conjugate 4 markedly extended survival compared to vehicle or 0.25 mg/kg Conjugate 4.

### Example 10

### Assessing the Efficacy of BCMA ADC Variants with Different Anti-BCMA Antibodies in Subcutaneous Arp-1 Tumors

This example evaluates the activity of BCMA ADC variants in subcutaneous ARP-1.

Female SCID beige mice 10 weeks of age were anesthetized with isoflurane and implanted subcutaneously into the right hind flank with a 1:1 mixture of 8×10⁶ human ARP-1 MM cells and matrigel. Randomization and start of treatment (Day 0 post treatment) was initiated when the average tumor size was approximately 150 mm³ (14 days post-implantation). The test articles and treatment groups are outlined in Table 12. All investigational test articles were formulated in 10 mM citrate pH 6.0, 10% sucrose. Body weight and tumor size were monitored at least 1-2×/week. Primary study endpoint was when the mean tumor size of the vehicle control group was >1,200 mm³.

TABLE 12
List of treatment groups

| | | Dose | Dosing | | |
|---|---|---|---|---|---|
| Group | Treatment | (mg/kg) | frequency | Route | N |
| 1 | PBS | — | single | IV | 8 |
| 2 | Conjugate 4 | 3 | single | IV | 8 |

Tumor size was analyzed using a one-way analysis of variance (ANOVA) with Dunnett's multiple comparison test. A probability of less than 5% (p<0.05) was considered statistically significant.

In this study, animals bearing established ARP-1 tumors were treated once with 3 mg/kg of BCMA ADC variants with different anti-BCMA antibodies and Conjugate 1. All test articles were well tolerated and did not exhibit any substantial toxicity defined as a >20% decrease in body weight.

Statistical analysis of tumor size on day 14 (when mean of the vehicle control tumors was >1,200 mm³) showed that all treatment groups were significantly efficacious compared to control. Conjugates 4 (˜70% TGI, p<0.001) was efficacious based on p values. Continued monitoring showed that Conjugates 4 was potent. Conjugate 1 was the most potent inducing tumor regression and stasis until ˜day 17.

### Example 11

### Assessing the Response of Subcutaneous ARP-1 Multiple Myeloma Tumors to Higher Doses of Conjugate 4

A study was conducted to assess the response of subcutaneous ARP-1 multiple myeloma tumors to higher doses of Conjugate 4.

Female severe combined immune deficient (SCID) Beige mice 9 weeks of age were anesthetized with isoflurane and implanted subcutaneously into the right hind flank with a 1:1 mixture of 1×10⁷ human ARP-1 MM cells and matrigel. Randomization and start of treatment (Day 0 post treatment) was initiated when the average tumor size was approximately 150 mm³ (14 days post-implantation). The treatment groups are outlined in Table 13. All Sutro investigational test articles were formulated in 10 mM citrate pH 6.0, 10% sucrose. Body weight and tumor size were monitored 1-2× per week. Primary study endpoint was when the mean tumor size of the vehicle control group was >1,500 mm³.

TABLE 13
List of Treatment Groups

| | | Dose | Dosing | | |
|---|---|---|---|---|---|
| Group | Treatment | (mg/kg) | frequency | Route | N |
| 1 | Vehicle | — | single | IV | 8 |
| 2 | Conjugate 4 | 5 | single | IV | 8 |
| 3 | Conjugate 4 | 10 | single | IV | 8 |
| 4 | Conjugate 4 | 15 | single | IV | 8 |
| 5 | Conjugate 4 | 20 | single | IV | 8 |
| 6 | Conjugate 1 | 5 | single | IV | 8 |

Body weight and tumor size were analyzed using a one-way analysis of variance (ANOVA) with Dunnett's multiple comparison test. A probability of less than 5% (p<0.05) was considered statistically significant.

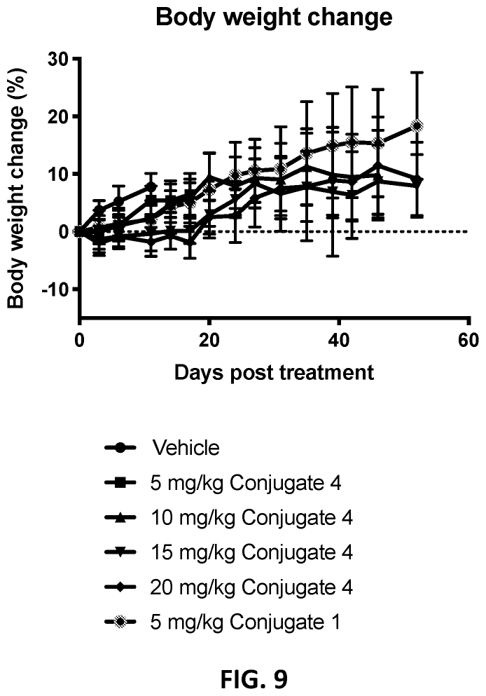

In this study, animals bearing established ARP-1 tumors were treated once with 4 dose levels of Conjugate 4 ranging from 5 to 20 mg/kg or 5 mg/kg of Conjugate 1. All test articles were well tolerated and none exhibited any toxicity based on body weight loss (FIG. 9). However, as the study progressed, an increase in body weight was observed in all the remaining treatment groups, with the most body weight change in animals treated with 5 mg/kg Conjugate 1. The continuous increase in body weight, as well as distended abdomens noted in some animals, suggested formation of internal ARP-1 tumors typically observed in this model. For this reason, the study was terminated on day 52.

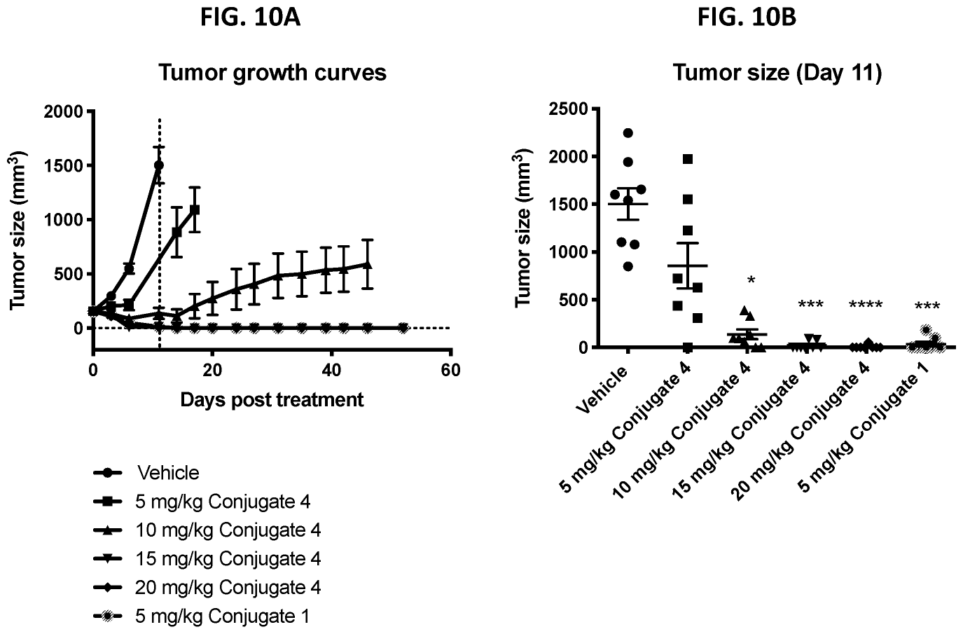

The effects of BCMA ADC Conjugate 4 and Conjugate 1 treatment on ARP-1 tumor growth are illustrated in FIGS. 10A and 10B. Increasing potency at escalating Conjugate 4 doses was observed indicating a linear dose-response relationship (FIG. 10A). Analysis of tumor size on day 11, when mean tumor size of the vehicle group reached study endpoint (>1,500 mm³), showed that Conjugate 4 exhibited significant efficacy compared to vehicle control starting at 10 mg/kg (FIG. 10B). Doses ≥10 mg/kg Conjugate 4 and 5 mg/kg Conjugate 1 induced tumor regression. Tumor re-growth for 4 out of 8 animals was seen starting at approximately day 11 for the 10 mg/kg Conjugate 4 group, while growth suppression was maintained up to day 52 for higher doses of Conjugate 4 or 5 mg/kg Conjugate 1 (FIG. 10A and FIG. 10B).

The results of this study show that Conjugate 4 at doses >15 mg/kg induced tumor regression and prolonged growth suppression for >50 days post treatment.

### Example 12

### Receptor Cross-Reactivity Analysis

The present example evaluates Conjugate 4 potential cross-reactive binding and recognition of human BCMA, BAFF-R and TACI receptors on engineered stable 293T cells. Results demonstrate that Conjugate 4 binds specifically to BMCA, but not to BAFF-R or TACI on engineered 293T cell lines. The control was Conjugate 1.

BCMA, B-cell activating factor receptor (BAFF-R, also referred to as TNFRSF13C) and transmembrane activator and calcium-modulator and cyclophilin ligand interactor (TACI, also referred to as TNFRSF13B) are homology-related type III transmembrane receptors with differential expression profiles and affinities for TNF (tumor necrosis factor) ligands, B-cell activating factor (BAFF, also referred to as BLyS) and a proliferation-inducing ligand (APRIL) to promote B cell survival and maturation (Hengeveld and Kerstan, 2015, Blood Cancer Journal 2015 Feb. 27; 5:e282).

293T cells were purchased from ATCC (American Type Culture Collection) and transfected with plasmids encoding human BCMA, BAFF-R and TACI using the Lipofectamine LTX Reagent with PLUS Reagent (ThermoFisher Scientific). Expression of human BCMA, BAFF-R and TACI on the stable cell lines were confirmed with commercial antibodies from BioLegend, anti-BCMA (clone 19F2), BAFF-R (clone 11C1) and TACI (clone 1A1).

Engineered 293T cells stably expressing human BCMA were treated with 1 μM DAPT, a secretase inhibitor (Santa Cruz Biotechnology), overnight prior to cell binding studies to maintain high level of BCMA expression. Parental and engineered 293T cells stably expressing BCMA, BAFF-R and TACI were collected, washed and resuspended in FACS buffer (DPBS buffer with 1% bovine serum albumin and 0.05% v/v sodium azide). Cells were plated in 96-well plates (100K per well) and incubated with Abs. Anti-human BCMA ADCs at 67 nM were incubated for 1 hour on ice. ADC binding was detected with phycoerythrin-conjugated anti-human Fc Ab (Jackson ImmunoResearch, West Grove, Pa.) for 1 hour on ice. Cells were analyzed using a BD FACS Canto system. FACS data were analyzed using Flowjo software to generate cell binding histograms.

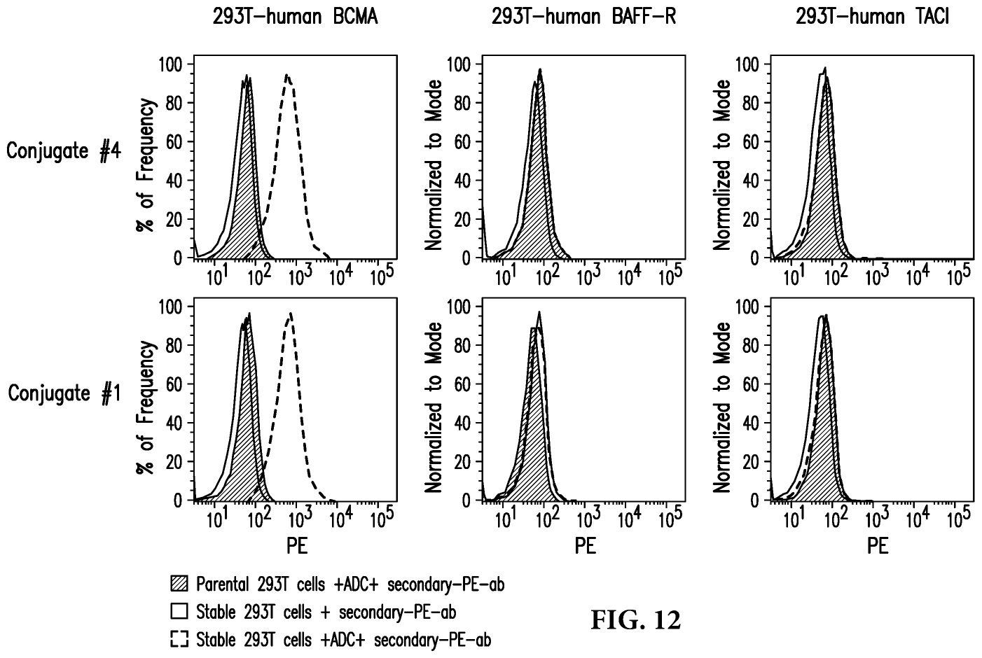

Both Conjugate 4 and the Conjugate 1 surrogate benchmark ADC, tested at a saturation concentration (67 nM), showed specific binding on 293T cells expressing human BCMA, but not BAFF-R and TACI (FIG. 12). These results indicated that Conjugate 4 binds specifically to BCMA, but not BAFF-R and TACI.

### Example 13

### In Vitro Cytotoxicity of Adcs Versus Free Drug Catabolites

The present example compares the relative cell killing activity of Conjugate 4 and Conjugate 1 (Maleimidocaproyl monomethylauristatin F) and their respective free-drug catabolites against a panel of different multiple myeloma cell lines.

Cytotoxic effects of ADCs and their respective free-drug catabolites were assessed in a tumor cell proliferation assay in two separate experiments. Twenty thousand cells per well were plated in 96-well flat-bottom half-area plates and ADC or free-drug catabolite was added to cells in cell culture media (n=3 replicates for each experiment) starting from 12.5 nM to 0.049 nM (2-fold dilutions) and from 2 μM to 0.03 nM for free-drug catabolites (4-fold dilutions). Cells were cultured at 37° C. in a CO₂ incubator for 3 days. For cell viability measurement, Cell TiterGlo® reagent (Promega Corp, Madison, Wis.) was added and plates were processed and read accordingly to the manufacturer's protocol. Relative luminescence was measured on an ENVISION® plate reader (Perkin-Elmer; Waltham, Mass.). Relative luminescence readings were converted to % viability using untreated cells as controls. Data was fitted with non-linear regression analysis, using log (inhibitor) vs. response, variable slope, 4-parameter fit equation using GraphPad Prism statistical software. Data was expressed as % viability relative to untreated control cells vs. dose of ADC in nM with error bars indicating the Standard Deviation (SD) of triplicates.

In two independent experiments, Conjugate 4 (Table 14) shows similar potent activity against three BCMA-positive MM cell lines (NCI-H929, OPM2 and U266B1) (Table 14) with EC₅₀ values ranging from 0.8 to 1.8 nM. In comparison, Conjugate 1, the J6M0-mcMMAF surrogate benchmark ADC (Table 14), shows slightly greater cell killing potency based on EC₅₀ values (0.2 to 0.9 nM), but with similar % span cell killing as Conjugate 4. Both ADCs do not show activity against the BCMA-negative K562 cell line.

The active catabolites of Conjugate 4 as free-drug compounds, 4-1 and 4-2 (Table 14), showed much weaker activity than the Conjugate 4 against all three BCMA-positive MM cell lines, including the BCMA-negative K562 cell line. In addition, the active catabolite of Conjugate 1 as a free-drug compound, 1-1 (Table 14), also showed weaker cell killing activity compared to Conjugate 4 on all four cell lines.

Data from these experiments indicate that anti-BCMA ADC Conjugate 4 is more potent than the released catabolite, which suggests that the cytotoxicity of Conjugate 4 is mainly due to BCMA-targeting and internalization in MM cells.

TABLE 14
In vitro cell-killing: ADCs and catabolites

| | NCI-H929 | | U266B1 | | OPM2 | | K562 | |
|---|---|---|---|---|---|---|---|---|
| ADC/ | EC₅₀ | Span | EC₅₀ | Span | EC₅₀ | Span | EC₅₀ | Span |
| catabolite | (nM) | (%) | (nM) | (%) | (nM) | (%) | (nM) | (%) |

Experiment 1

| | | | | | | | | |
|---|---|---|---|---|---|---|---|---|
| Conjugate 4 | 0.8 | 85 | 0.8 | 80 | 1.7 | 91 | NK | NK |
| Catabolite | 135 | 86 | 61 | 84 | 183 | 97 | NC | NC |
| 4-1 | | | | | | | | |
| Catabolite | 73 | 90 | 59 | 85 | 165 | 97 | NC | NC |
| 4-2 | | | | | | | | |
| Conjugate 1 | 0.2 | 85 | 0.2 | 85 | 0.8 | 95 | NK | NK |
| Catabolite | 398* | 100* | 106 | 94 | 480* | 100* | NC | NC |
| 1-1 | | | | | | | | |

Experiment 2

| | | | | | | | | |
|---|---|---|---|---|---|---|---|---|
| Conjugate 4 | 0.8 | 85 | 0.9 | 81 | 1.8 | 90 | NK | NK |
| Catabolite | 86 | 83 | 39 | 82 | 161 | 97 | NC | NC |
| 4-1 | | | | | | | | |
| Catabolite | 70 | 92 | 54 | 85 | 159 | 96 | NC | NC |
| 4-2 | | | | | | | | |
| Conjugate 1 | 0.2 | 86 | 0.3 | 84 | 0.9 | 94 | NK | NK |
| Catabolite | 393* | 94* | 110 | 89 | 514* | 100* | NC | NC |
| 1-1 | | | | | | | | |

*Estimated value
NC: Not calculable due to incomplete dilution curve
NK: No killing observed
ADC: Antibody drug conjugate

### Example 14

### In Vitro Cytotoxicity Comparison on Multiple Myeloma Cell Lines Versus GFP Control

The present example evaluates the cell killing activity of Conjugate 4 compared to the respective anti-GFP negative control conjugate Conjugate 20 at DAR4 on three BCMA-positive MM cell lines (NCI-H929, U266B1 and OPM-2) and one BCMA-negative cell line (K562).

As a negative control ADC for this experiment, an anti-GFP IgG was generated as a cell free (CF)-produced antibody. The antibody was conjugated to the same drug linker, see Conjugate M, at the same Y180 and F404 sites on the anti-GFP heavy chain to yield Conjugate 20.

Cytotoxic effects of Conjugate 4 and the respective anti-GFP negative control ADC, Conjugate 20, were assessed in a tumor cell proliferation assay in two separate experiments. In both experiments, Conjugate 4 showed potent cell killing activity on all three BCMA-positive MM cell lines (NCI-H929, OPM-2 and U266B1) with EC₅₀ values ranging from 0.7 to 2.0 nM (Table 15). No cell killing was observed for Conjugate 4 on the BCMA-negative K562 cell line. In comparison, the anti-GFP Conjugate 20 negative control ADC did not show any cell killing activity against any of the four cell lines tested. Data from these experiments suggests that the in vitro cell killing effect of Conjugate 4 is mediated through BCMA-target mediated internalization of the ADC in BCMA-positive MM cell lines.

TABLE 15
Summary of Cell Killing EC₅₀ and Span Against Different Cell Lines

| | NCI-H929 | | OPM2 | | U266B1 | | K562 | |
|---|---|---|---|---|---|---|---|---|
| Conjugate | EC₅₀ | Span | EC₅₀ | Span | EC₅₀ | Span | EC₅₀ | Span |
| No. | (nM) | (%) | (nM) | (%) | (nM) | (%) | (nM) | (%) |

Experiment 1

| | | | | | | | | |
|---|---|---|---|---|---|---|---|---|
| 4 | 0.7 | 89 | 1.7 | 87 | 0.7 | 78 | NK | NK |

Experiment 2

| | | | | | | | | |
|---|---|---|---|---|---|---|---|---|
| 4 | 0.8 | 89 | 2 | 88 | 0.8 | 79 | NK | NK |

NK = No Killing

### Example 15

### Specificity of Conjugate Cell Killing Activity

The example evaluates the specific cell killing activity of Conjugate 4 for BCMA-expressing multiple myeloma cells.

Cytotoxic effects of ADCs (Conjugate 4, Conjugate 1) in the absence or presence of excess unconjugated anti-BCMA antibody, 2265-F02, and recombinant human BCMA Extra Cellular Domain (ECD) protein (catalog 310-16, PeproTech, NJ, USA) were assessed in a tumor cell proliferation assay. Twenty thousand cells per well were plated in 96-well flat-bottom half-area plates. Recombinant human BCMA ECD protein at 2 μM concentration (100-fold excess of the highest ADC concentration) was pre-incubated with ADCs for 1 hour at room temperature prior to adding it to cells to block the BCMA binding sites on the ADCs. Unconjugated anti-BCMA antibody, 2265-F02, was added to cells at 500 nM concentration (25-fold excess of the highest ADC concentration) for 1 hour at room temperature. 2-fold serial dilutions of ADCs were then added into the well with the starting concentration of 20 nM and the final concentration of 0.078 nM. Cells were cultured at 37° C. in a CO₂ incubator for 3 days. For cell viability measurement, Cell Titer-Glo® reagent (Promega Corp, Madison, Wis.) was added and plates were processed and read accordingly to the manufacturer's protocol. Relative luminescence was measured on an ENVISION® plate reader (Perkin-Elmer; Waltham, Mass.). Relative luminescence readings were converted to % viability using untreated cells as controls. Data (mean of the duplicates) was fitted with non-linear regression analysis, using log (inhibitor) vs. response, variable slope, 4-parameter fit equation using GraphPad Prism statistical software. Data was plotted as % of cell viability relative to untreated control well vs. dose of ADC in nanomolar (nM) with error bars indicating the Standard Deviation (SD) of duplicates.

Conjugate 4 and Conjugate 1 surrogate benchmark ADC (Table 16) showed potent cell killing activity on all four BCMA-positive MM cell lines tested (Table 16) with EC₅₀ values ranging from 0.4 to 3.3 nM (Table 16). No cell killing was observed for Conjugate 4 or Conjugate 1 in the presence of excess unconjugated anti-BCMA Ab, 2265-F02, or recombinant human BCMA ECD protein across all four BCMA-positive cell lines. Data from this experiment indicates that the in vitro cell killing effect of Conjugate 4 is specific for BCMA.

TABLE 16
Summary of Cell Killing EC₅₀ and Span Against Different Cell Lines

| | | NCI-H929 | | OPM2 | | U266B1 | | ARP-1 | |
|---|---|---|---|---|---|---|---|---|---|
| Conjugate | Competing | EC₅₀ | Span | EC₅₀ | Span | EC₅₀ | Span | EC₅₀ | Span |
| No. | Reagent | (nM) | (%) | (nM) | (%) | (nM) | (%) | (nM) | (%) |
| 4 | none | 0.9 | 85 | 3.3 | 85 | 0.9 | 84 | 0.7 | 95 |
| | 2 μM BCMA ECD | NK | NK | NK | NK | NK | NK | NK | NK |
| | 0.5 μM 2265-F02 | NK | NK | NK | NK | NK | NK | NK | NK |
| 1 | none | 0.4 | 86 | 2.3 | 92 | 0.4 | 90 | 0.6 | 97 |
| | 2 μM BCMA ECD | NK | NK | NK | NK | NK | NK | NK | NK |

NK = No Killing

### Example 16

### In Vitro Cell Binding and Cell Killing: Multiple Myeloma Cell Lines

This example compares in vitro cell binding and cell killing potency of Conjugate 4 versus the Conjugate 1 (Maleimidocaproyl monomethylauristatin F) surrogate benchmark ADC across a large panel of multiple myeloma (MM) cell lines expressing BCMA. In this experiment, Conjugate 4 shows better cell binding and similar potent cell killing compared to the surrogate benchmark ADC.

NCI-H929, U266B1, RPMI-8226, MM.1S, MC/CAR and K-562 cells were purchased from ATCC (American Type Culture Collection, Manassas, Va., USA). OPM-2 cells were purchased from The Leibniz Institute DSMZ (German Collection of Microorganisms and Cell Cultures GmbH, Braunschweig, Germany). ARP-1 cells were liscensed from the laboratory of Dr. Jonathan J. Keats from the Translational Genomics Research Institute (Phoenix, Ariz., USA). All cell lines were maintained in RPMI high glucose media (Corning, Corning, N.Y.) supplemented with 20% heat-inactivated fetal bovine serum (Thermo Scientific, Grand Island, N.Y.), 2 mM glutamax (Thermo Scientific, Grand Island, N.Y.), and 1× Penicillin/streptomycin (Corning, Corning, N.Y.).

Tumor cells were collected, washed and resuspended in FACS buffer (DPBS buffer with 1% bovine serum albumin and 0.05% v/v sodium azide). MM cells pre-incubated with 2.5 μg of Human Fc Block (BD Biosciences, cat 564220) for 10 minutes at room temperature were plated in 96-well plates (100-200K per well) and incubated with antibodies (titrated from 66.7 nM with 3-fold serial dilutions) for 1 hour on ice. Antibody binding was detected with phycoerythrin-conjugated anti-human Fc Ab (Jackson ImmunoResearch, West Grove, Pa.) for 1 hour on ice. Cells were analyzed using a BD FACS Canto system. Fluorescence-activated cell sorting (FACS) data were analyzed using Flowjo software to calculate mean fluorescence intensity (MFI) (n=3 replicates) and data (mean MFI+/−standard error of the mean [SEM] versus nM of the antibody) was generated using the GraphPad Prism software.

Cytotoxic effects of Conjugate 4, 2265-F02 (as the negative unconjugated antibody version of Conjugate 4) and the Conjugate 1 surrogate benchmark ADC were assessed in a tumor cell proliferation assay.

Both Conjugate 4 and its unconjugated antibody version, 2265-F02, showed similarly high affinity binding on six MM cell lines (NCI-H929, ARP-1, OPM-2, U266B1, MM.1S and RPMI-8226) with K_{D} ranging from 0.9 to 3.9 nM. In comparison, the Conjugate 1 mcMMAF surrogate benchmark ADC showed weaker binding. The binding curves for 2265-F02 were not saturated at 66.7 nM. All three Abs tested showed no significant binding on BCMA-negative myeloma MC/CAR cells (Table 17). Results indicate that drug-linker conjugation on F404/Y180 sites does not affect binding of the anti-BCMA antibody and that Conjugate 4 ADC has high affinity binding for BCMA-expressing MM cell lines.

Both Conjugate 4 and Conjugate 1 surrogate benchmark ADCs showed similar potent cell killing activity across five of the six MM cell lines expressing BCMA. Cell killing potency EC₅₀ ranged from 0.70 to 2.1 for Conjugate 4 ADC and 0.29 to 1.4 nM for Conjugate 1 surrogate benchmark ADC, respectively (Table 18). Low cell killing activity was observed for both ADCs on the low BCMA-expressing RPMI-8226 MM cell line. Results indicate that Conjugate 4 has potent cell killing potential against multiple MM cell lines.

Conjugate 4 binds to BCMA-expressing MM cell lines with high affinity and shows potent cell killing activity, similar to the Conjugate 1 surrogate benchmark ADC, across five of the six MM cell lines expressing BCMA.

TABLE 17
Summary of K_{D} and Bₘₐₓ Binding on Different MM Cell Lines

| | | 2265-F02 | | Conjugate 4 | | Conjugate 1 | |
|---|---|---|---|---|---|---|---|
| | BCMA | K_{D} | | K_{D} | | K_{D} | |
| Cell Line | Copy#/Cell | (nM) | Bmax | (nM) | Bmax | (nM) | Bmax |
| NCI-H929 | 171,234 | 0.9 | 450 | 2.4 | 469 | NC | NC |
| ARP-1 | 47,937 | 1 | 112 | 1.2 | 115 | 2.4 | 45 |
| OPM-2 | 47,221 | 1.3 | 389 | 2.3 | 381 | NC | NC |
| U266B1 | 29,649 | 1.4 | 151 | 3.9 | 133 | NC | NC |
| MM.1S | 21,447 | 0.9 | 162 | 1.6 | 141 | NC | NC |
| RPMI-8226 | 20,640 | 1.3 | 193 | 1.8 | 249 | NC | NC |
| MC/CAR | <LOD | NSB | NSB | NSB | NSB | NSB | NSB |

<LOD = Below limit of detection
NC = Binding observed, but K_{D} and Bmax Not Calculable due to incomplete dilution curve
NSB = No significant binding

TABLE 18
Summary of EC₅₀ and Cell Killing Span on Different MM Cell Lines

| | | 2265-F02 | | Conjugate 4 | | Conjugate 1 | |
|---|---|---|---|---|---|---|---|
| | BCMA | EC₅₀ | Span | EC₅₀ | Span | EC₅₀ | Span |
| Cell Line | Copy#/Cell | (nM) | (%) | (nM) | (%) | (nM) | (%) |
| NCI-H929 | 171,234 | NK | NK | 0.8 | 89 | 0.29 | 91 |
| ARP-1 | 47,937 | NK | NK | 0.7 | 95 | 0.52 | 95 |
| OPM-2 | 47,221 | NK | NK | 2.1 | 88 | 1.4 | 93 |
| U266B1 | 29,649 | NK | NK | 0.86 | 84 | 0.32 | 90 |
| MM.1S | 21,447 | NK | NK | 0.82 | 77 | 0.68 | 86 |
| RPMI-8226 | 20,640 | NK | NK | NC | 15 | NC | 40 |
| MC/CAR | <LOD | NK | NK | NK | NK | NK | NK |
| K-562 | <LOD | NK | NK | NK | NK | NK | NK |

<LOD = Below limit of detection
NC = Cell killing observed, but EC₅₀ and span Not Calculable due to imcomplete dilution curve
NK = No Killing

### Example 17

### In Vitro Cell Binding and Cell Killing: Species Cross-Reactivity

This example compares in vitro cell binding and cell killing potency of Conjugate 4 versus the Conjugate 1 (Maleimidocaproyl monomethylauristatin F) surrogate benchmark ADC on stable 293T cells overexpressing human, cynomolgus primate, rat, or mouse BCMA.

293T cells were purchased from ATCC (American Type Culture Collection) and transfected with plasmids encoding human, cynomolgus primate or rat BCMA using the Lipofectamine LTX Reagent with PLUS Reagent (ThermoFisher Scientific). 293T-mouse BCMA cells were generated by transfecting HEK293T cells with plasmids encoding mouse BCMA (Invivogen) using FUGENE HD reagent (Promega).

Engineered 293T cells stably expressing human, cynomolgus primate or rat BCMA were treated with 1 μM DAPT, a γ-secretase inhibitor (Santa Cruz Biotechnology), overnight prior to cell binding studies to maintain high level of BCMA expression. Cells were collected, washed and resuspended in FACS buffer (DPBS buffer with 1% bovine serum albumin and 0.05% v/v sodium azide). Cells were plated in 96-well plates (100K per well) and incubated with Abs (titrated from 200 nM with 2-fold serial dilutions) for 1 hour on ice. Ab binding was detected with phycoerythrin-conjugated anti-human Fc Ab (Jackson ImmunoResearch, West Grove, Pa.) for 1 hour on ice. Cells were analyzed using a BD FACS Canto system.

293T-mouse BCMA cells were collected, washed and suspended in FACS buffer (DPBS buffer with 1% bovine serum albumin and 0.05% v/v sodium azide). Cells were plated in 96-well plates (100 k per well) and incubated with antibodies (titrated half-log serial dilutions from 200 nM) for 1 hour on ice. Cells were washed then antibody binding was detected with phycoerythrin-conjugated anti-human Fc secondary antibody (Jackson ImmunoResearch, West Grove, Pa.) for 1 hour on ice. Cells were analyzed using a BD LSR-Fortessa X-20 flow cytometry system. FACS data were analyzed using Flowjo software to calculate geometric fluorescence intensity (gMFI) (n=3 replicates) and data (geo. Mean MFI+/−SEM versus log nM Ab) were generated using GraphPad Prism software.

Cytotoxic effects of SP8919 ADC and the J6M0-mcMMAF surrogate benchmark ADC were assessed in a tumor cell proliferation assay. 500 cells per well were plated in 96-well flat-bottom half-area plates overnight and ADCs were added to cells the next day in cell culture media (n=3 replicates) starting at 20 nM (2-fold dilutions). Cells were cultured at 37° C. in a CO₂ incubator for 5 days. For cell viability measurement, Cell Titer-Glo® reagent (Promega Corp, Madison, Wis.) was added and plates were processed and read accordingly to the manufacturer's protocol. Relative luminescence was measured on an ENVISION® plate reader (Perkin-Elmer; Waltham, Mass.). Relative luminescence readings were converted to % viability using untreated cells as controls. Data was fitted with non-linear regression analysis, using log (inhibitor) vs. response, variable slope, 4-parameter fit equation using GraphPad Prism statistical software. Data was expressed as % relative cell viability vs. dose of ADC (mean+/−SEM).

Both Conjugate 4 and its unconjugated Ab version, 2265-F02 Y180/F404, showed similarly high affinity binding on 293T cells overexpressing human and cynomolgus, but not parental 293T cells or cells stably transfected to express rat BCMA or mouse BCMA. K_{d} binding to human and cynomolgus BCMA-expressing 293T cells ranged from 1.4 to 2.8 nM (Table 19). In comparison, the Conjugate 1 surrogate benchmark ADC showed slightly weaker binding activity with K_{d} values ranging from 7.1 to 8.6 nM (Table 19). Results indicate that linker payload conjugation at F404/Y180 sites does not affect binding of the anti-BCMA Conjugate 4 compared to the unconjugated Ab control and that Conjugate 4 binds to human and cynomolgus primate BCMA, but not rat or mouse BCMA.

Based on the positive species cross-reactive cell binding results, cell killing activity of Conjugate 4 and the Conjugate 1 surrogate benchmark ADC was compared on 293T cells expressing human or cynomolgus primate BCMA. Both Conjugate 4 and the Conjugate 1 surrogate benchmark ADCs showed similar cell killing activity on stably-transfected 293T cells expressing human and cynomolgus primate BCMA, but not parental 293T cells. Results indicate that Conjugate 4 has cynomolgus primate BMCA binding reactivity similar to the Conjugate 1 surrogate benchmark ADC, which was confirmed by the cell killing assay.

Overall, results from this experiment indicates that Conjugate 1 and Conjugate 4 showed specific cell binding recognition and cell killing sensitivity against 293T cells overexpressing human and cynomolgus primate BCMA but did not bind rat or mouse BCMA. This suggests that similar to the Conjugate 1 surrogate benchmark ADC, Conjugate 4 can be tested for toxicity assessment in cynomolgus primates.

TABLE 19
Summary of K_{d} and Bₘₐₓ Binding on 293T Cells Stably Expressing Human,
Cynomolgus Primate, Rat or Mouse BCMA

2265-F02 Y180/F404

| | (unconjugated Ab) | | Conjugate 4 | | Conjugate 1 | |
|---|---|---|---|---|---|---|
| | Kd | | Kd | | Kd | |
| Cell Line | (nM) | Bmax | (nM) | Bmax | nM) | Bmax |
| 293T | NB | NB | NB | NB | NB | NB |
| 293T-hBCMA | 2.1 | 1186 | 1.4 | 994 | 7.1 | 856 |
| 293T-cBCMA | 2.8 | 1104 | 2.7 | 913 | 8.6 | 981 |
| 293T-rBCMA | NB | NB | NB | NB | NB | NB |
| 293T-mBCMA | NB | NB | NB | NB | NB | NB |

hBCMA: human BCMA, cBCMA: cynomolgous BCMA, rBCMA: rat BCMA, mBCMA: mouse BCMA, NB: No binding

### Example 18

### ADC Blockade of BCMA Binding to BAFF and April Ligands

This example compares Conjugate 4 ADC and the Conjugate 1 surrogate benchmark ADC in blocking BCMA receptor binding to ligands BAFF (B cell activating factor) and APRIL (a proliferation inducing ligand).

BCMA binds to ligands, BAFF and APRIL to mediate survival of bone marrow plasma cells and plasmablasts, as well as MM cell growth and survival. Tai et al., 2014, Blood I23(20):3128-38. The J6M0 Ab was reported to block BAFF and APRIL binding as an additional therapeutic mechanism of action, in addition to being an ADC to target BCMA-expressing MM cells. Tai et al., supra.

Recombinant human BCMA ECD protein (Acro Biosystems) was coated at 0.5 μg/ml in carbonate/bicarbonate pH 9.6 buffer (Sigma-Aldrich) overnight at 4° C. in 96-well Nunc MaxiSorp plates. All following steps were performed at room temperature. Plates were washed with PBST buffer (DPBS+0.05% Tween-20) and blocked with ELISA blocking buffer (DPBS+1% BSA) for 1 hour. Abs and ligands were diluted in ELISA diluent buffer (DPBS+0.5% BSA+0.05% Tween-20) and mixed in a 1:1 volume ratio starting at a final concentration of 200 nM with two-fold serial dilutions for test Abs with recombinant ligands, BAFF or APRIL, at 1 ng/ml and 10 ng/ml final concentrations, respectively. Mixed Ab and ligand was added to human BCMA coated plates for binding for 2 hours. Plates were washed and streptavidin-conjugated HRP Ab (Jackson ImmunoResearch) was diluted 1,000-fold in ELISA diluent buffer and added to plates for 1 hour in the dark. Plates were washed and TMB substrate (SureBlue Reserve, KPL) was added for 20 minutes in the dark. Substrate reaction was quenched with an equal volume of 1M phosphoric acid and plates were read at 450 nm on the M5 SpectraMax plate reader (Molecular Devices). OD values were plotted and GraphPad Prism software was used to create one site, specific binding with Hill slope curves (log transform) to determine IC₅₀ values (mean±SEM, n=2).

Both Conjugate 4 ADC and the Conjugate 1 surrogate benchmark ADC showed equivalent activity in blocking both BAFF (Table 20) and APRIL (Table 21) ligand binding to recombinant BCMA by ELISA with IC₅₀ values ranging from 6.8 to 8.9 nM. Anti-Her2 antibody Trastuzumab was added as negative control in the assays and did not block BAFF nor APRIL binding to BCMA.

Results indicate that Conjugate 4 ADC blocks both BAFF and APRIL ligand binding to BCMA and suggest that Conjugate 4 ADC may share the same additional mechanism of action as Conjugate 1 in potentially reducing MM cell proliferation.

TABLE 20
Summary BAFF IC50

| | Experiment No. 1 | Experiment No. 2 |
|---|---|---|
| Conjugate No. | IC50 (nM) | IC50 (nM) |
| Conjugate 4 | 6.8 | 6.9 |
| Conjugate 1 | 7 | 7.4 |

TABLE 21
Summary APRIL IC50

| | Experiment No. 1 | Experiment No. 2 |
|---|---|---|
| Conjugate No. | IC50 (nM) | IC50 (nM) |
| Conjugate 4 | 8.3 | 8.9 |
| Conjugate 1 | 6.8 | 8.5 |

### Example 19

### Chemical Characteristics of Conjugate 4

Conjugate 4 is a conjugate of antibody and drug-linker. Conjugate 4 is an aglycosylated anti-B-cell maturation antigen (anti-BCMA) humanized IgG1 antibody drug conjugate (ADC) comprised of an anti-BCMA IgG1 humanized antibody (aglycosylated 2265-F02) conjugated covalently at the non-natural amino acid (nnAA) para-azidomethyl-L-phenylalanine (pAMF) residue at nominal positions 180 and 404 by EU numbering (actual positions 186 and 410) to a 20-methyl-1-(3-methyl-3,9-dihydro-8Hdibenzo[b,f][1,2,3]triazolo[4,5-d]azocin-8-yl)-1,5,21-trioxo-8,11,14,17-tetraoxa-4,20-diazapentacosan-25-oyl (desacetyl) maytansinoid drug-linker. The ADC, Conjugate 4, is a single predominant conjugated species (existing as a ˜1:1 mixture of two regioisomers) with a drug to antibody ratio (DAR) of 4. The molecular weight of Conjugate 4 is approximately 151 kDa. A sample of Conjugate 4, prepared using the methods described herein, exhibited a DAR of 3.9 to 4, as measured and calculated using the methods described herein (see, e.g., Example 6).

Disulfide bonds in Conjugate 4 are as follows: Inter chain (LC1): Cys 24-Cys 89; Cys 135-Cys 195. Inter Chain (HC1): Cys 23-Cys 97; Cys 150-Cys 206; Cys 267-Cys 327; Cys 373-Cys 431. Inter Chain (HC2): Cys 23-Cys 97; Cys 150-Cys 206; Cys 267-Cys 327; Cys 373-Cys 431. Inter chain (LC2): Cys 24-Cys 89; Cys 135-Cys 195. Intra-LC1-HC-1: Cys 215-Cys 226. Intra-LC2-HC-2: Cys 215-Cys 226. Intra-HC-HC-Hinge-1: Cys 232-Cys 232. Intra-HC-HC-Hinge-2: Cys 235-Cys 235.

### Example 20

### Sequences

Table 22 provides sequences referred to herein.

TABLE 22
Sequences

| SEQ | | | | |
|---|---|---|---|---|
| ID | | | | |
| NO: | Molecule | Region | Scheme | Sequence |
| 1 | Human BCMA | | | MLQMAGQCSQNEYFD |
| | (Isoform 1, | | | SLLHACIPCQLRCSS |
| | UniprotKB- | | | NTPPLTCQRYCNASV |
| | Q02223) | | | TNSVKGTNAILWTCL |
| | | | | GLSLIISLAVFVLMF |
| | | | | LLRKINSEPLKDEFK |
| | | | | NTGSGLLGMANIDLE |
| | | | | KSRTGDEIILPRGLE |
| | | | | YTVEECTCEDCIKSK |
| | | | | PKVDSDHCFPLPAME |
| | | | | EGATILVTTKTNDYC |
| | | | | KSLPAALSATEIEKS |
| | | | | ISAR |
| 2 | Human BCMA | | | MLQMAGQCSQNEYFD |
| | (Isoform 2, | | | SLLHACIPCQLRCSS |
| | UniprotKB- | | | NTPPLTCQRYCNARS |
| | Q02223) | | | GLLGMANIDLEKSRT |
| | | | | GDEIILPRGLEYTVE |
| | | | | ECTCEDCIKSKPKVD |
| | | | | SDHCFPLPAMEEGAT |
| | | | | ILVTTKTNDYCKSLP |
| | | | | AALSATEIEKSISAR |
| 3 | Cynomolgus | | | MLQMARQCSQNEYFD |
| | BCMA (Predicted | | | SLLHDCKPCQLRCSS |
| | NCBI Reference | | | TPPLTCQRYCNASMT |
| | Sequence: | | | NSVKGMNAILWTCLG |
| | XP_001106892.1) | | | LSLIISLAVFVLTFL |
| | | | | LRKMSSEPLKDEFKN |
| | | | | TGSGLLGMANIDLEK |
| | | | | GRTGDEIVLPRGLEY |
| | | | | TVEECTCEDCIKNKP |
| | | | | KVDSDHCFPLPAMEE |
| | | | | GATILVTTKTNDYCN |
| | | | | SLSAALSVTEIEKSI |
| | | | | SAR |
| 4 | Murine BCMA | | | MAQQCFHSEYFDSLL |
| | (NBCI Reference | | | HACKPCHLRCSNPPA |
| | Sequence: | | | TCQPYCDPSVTSSVK |
| | NP_035738.1) | | | GTYTVLWIFLGLTLV |
| | | | | LSLALFTISFLLRKM |
| | | | | NPEALKDEPQSPGQL |
| | | | | DGSAQLDKADTELTR |
| | | | | IRAGDDRIFPRSLEY |
| | | | | TVEECTCEDCVKSKP |
| | | | | KGDSDHFFPLPAMEE |
| | | | | GATILVTTKTGDYGK |
| | | | | SSVPTALQSVMGMEK |
| | | | | PTHTR |
| 5 | 2265-F02 | CDR-H1 | Chothia | GFNISAP |
| 6 | 2265-F02 | CDR-H1 | Kabat | APGIH |
| 7 | 2265-F02 | CDR-H2 | Chothia | NPAGGY |
| 8 | 2265-F02 | CDR-H2 | Kabat | FINPAGGYTDYADSV |
| | | | | KG |
| 9 | 2265-F02 | CDR-H3 | | DYIRQYWTYVLDY |
| 10 | trastuzumab | CDR-L1 | | RASQDVNTAVA |
| 11 | trastuzumab | CDR-L2 | | SASFLYS |
| 12 | trastuzumab | CDR-L3 | | OOHYTTPPT |
| 13 | 2265-F02 | V_{H} | | EVQLVESGGGLVQPG |
| | | | | GSLRLSCAASGFNIS |
| | | | | APGIHWVRQAPGKGL |
| | | | | EWVGFINPAGGYTDY |
| | | | | ADSVKGRFTISADTS |
| | | | | KNTAYLQMNSLRAED |
| | | | | TAVYYCARDYIRQYW |
| | | | | TYVLDYWGOGTLVTV |
| | | | | SS |
| 14 | trastuzumab | V_{L} | | DIQMTQSPSSLSASV |
| | | | | GDRVTITCRA |
| | | | | SQDVNTAVAWYQQKP |
| | | | | GKAPKLLI |
| | | | | YSASFLYSGVPSRFS |
| | | | | GSRSGTDFTL |
| | | | | TISSLQPEDFATYYC |
| | | | | QQHYTTPPTF |
| | | | | GQGTKVEIK |
| 15 | Antibody 2265- | Heavy | | EVQLVESGGGLVQPG |
| | F02 | Chain | | GSLRLSCAASGFNIS |
| | | | | APGIHWVRQAPGKGL |
| | | | | EWVGFINPAGGYTDY |
| | | | | ADSVKGRFTISADTS |
| | | | | KNTAYLQMNSLRAED |
| | | | | TAVYYCARDYIRQYW |
| | | | | TYVLDYWGQGTLVTV |
| | | | | SSASTKGPSVFPLAP |
| | | | | SSKSTSGGTAALGCL |
| | | | | VKDYFPEPVTVSWNS |
| | | | | GALTSGVHTFPAVLQ |
| | | | | SSGLYSLSSVVTVPS |
| | | | | SSLGTQTYICNVNHK |
| | | | | PSNTKVDKKVEPKSC |
| | | | | DKTHTCPPCPAPELL |
| | | | | GGPSVFLFPPKPKDT |
| | | | | LMISRTPEVTCVVVD |
| | | | | VSHEDPEVKFNWYVD |
| | | | | GVEVHNAKTKPREEQ |
| | | | | YNSTYRVVSVLTVLH |
| | | | | QDWLNGKEYKCKVSN |
| | | | | KALPAPIEKTISKAK |
| | | | | GQPREPQVYTLPPSR |
| | | | | EEMTKNQVSLTCLVK |
| | | | | GFYPSDIAVEWESNG |
| | | | | QPENNYKTTPPVLDS |
| | | | | DGSFFLYSKLTVDKS |
| | | | | RWQQGNVFSCSVMHE |
| | | | | ALHNHYTQKSLSLSP |
| | | | | GK |
| | | | | Residues in |
| | | | | bold are |
| | | | | replaced with |
| | | | | p-azidomethyl- |
| | | | | phenylalanine |
| | | | | in Antibody |
| | | | | 2265-F02. |
| 16 | Antibody 2265- | Heavy | | GAAGTTCAGTTAGTG |
| | F02 | Chain | | GAATCAGGCGGCGGT |
| | | | | TTAGTTCAACCAGGC |
| | | | | GGTTCATTGCGTCTG |
| | | | | TCATGCGCGGCTTCC |
| | | | | GGTTTCAACATCAGT |
| | | | | GCGCCTGGGATCCAT |
| | | | | TGGGTGCGTCAGGCC |
| | | | | CCAGGCAAGGGTCTG |
| | | | | GAGTGGGTCGGTTTT |
| | | | | ATCAATCCTGCTGGC |
| | | | | GGTTATACCGACTAT |
| | | | | GCGGACTCTGTGAAG |
| | | | | GGTCGCTTCACCATT |
| | | | | AGCGCGGATACCTCG |
| | | | | AAGAATACGGCGTAT |
| | | | | TTACAGATGAATTCC |
| | | | | CTGCGTGCAGAGGAC |
| | | | | ACTGCCGTCTACTAT |
| | | | | TGTGCGCGCGATTAC |
| | | | | ATTCGGCAGTACTGG |
| | | | | ACCTACGTTCTTGAC |
| | | | | TACTGGGGCCAGGGT |
| | | | | ACGCTGGTCACCGTG |
| | | | | TCGTCGGCGTCAACC |
| | | | | AAGGGTCCGTCGGTT |
| | | | | TTTCCGCTGGCGCCG |
| | | | | TCGTCAAAATCTACG |
| | | | | TCCGGTGGTACCGCC |
| | | | | GCTCTGGGTTGCCTG |
| | | | | GTTAAAGACTACTTT |
| | | | | CCGGAGCCGGTCACG |
| | | | | GTTTCGTGGAACTCT |
| | | | | GGTGCCCTGACTTCT |
| | | | | GGCGTCCACACGTTC |
| | | | | CCAGCCGTTTTGCAG |
| | | | | TCATCCGGTCTGTAG |
| | | | | TCGTTGTCCTCTGTG |
| | | | | GTCACGGTGCCGTCA |
| | | | | TCGTCTCTGGGCACC |
| | | | | CAAACCTATATCTGC |
| | | | | AATGTCAACCACAAA |
| | | | | CCGTCCAATACGAAA |
| | | | | GTTGACAAAAAAGTC |
| | | | | GAGCCGAAATCTTGC |
| | | | | GACAAGACCCACACG |
| | | | | TGCCCTCCGTGCCCG |
| | | | | GCACCGGAACTGCTG |
| | | | | GGCGGTCCGTCGGTG |
| | | | | TTCCTGTTCCCGCCG |
| | | | | AAGCCGAAAGATACT |
| | | | | CTGATGATCTCACGT |
| | | | | ACCCCGGAAGTCACG |
| | | | | TGTGTTGTTGTTGAC |
| | | | | GTGTCACACGAAGAT |
| | | | | CCAGAGGTGAAATTC |
| | | | | AATTGGTATGTGGAC |
| | | | | GGTGTCGAAGTGCAT |
| | | | | AATGCCAAAACCAAA |
| | | | | CCGCGCGAGGAACAG |
| | | | | TACAACTCCACCTAC |
| | | | | CGCGTCGTGTCGGTG |
| | | | | TTGACCGTCCTGCAT |
| | | | | CAAGACTGGCTGAAC |
| | | | | GGTAAAGAGTACAAG |
| | | | | TGCAAGGTTTCAAAT |
| | | | | AAGGCACTGCCTGCG |
| | | | | CCGATTGAAAAGACC |
| | | | | ATCTCTAAGGCAAAG |
| | | | | GGCCAGCCGCGTGAG |
| | | | | CCACAGGTGTATACC |
| | | | | CTGCCGCCGTCGCGT |
| | | | | GAAGAAATGACCAAG |
| | | | | AACCAAGTTTCACTG |
| | | | | ACGTGTCTGGTCAAG |
| | | | | GGCTTTTATCCGTCC |
| | | | | GATATTGCGGTGGAG |
| | | | | TGGGAGTCTAATGGC |
| | | | | CAGCCGGAAAACAAT |
| | | | | TACAAAACGACTCCG |
| | | | | CCGGTGCTGGATTCC |
| | | | | GACGGTTCGTAGTTC |
| | | | | CTGTATTCCAAGCTG |
| | | | | ACCGTTGACAAATCA |
| | | | | CGTTGGCAGCAAGGC |
| | | | | AACGTTTTTTCTTGT |
| | | | | TCGGTAATGCACGAA |
| | | | | GCGCTGCACAATCAT |
| | | | | TACACCCAGAAATCA |
| | | | | CTGTCGTTGTCTCCG |
| | | | | GGCAAA |
| 17 | Antibody 2265- | Light | | DIQMTQSPSSLSASV |
| | F02 | Chain | | GDRVTITCRASQDVN |
| | | | | TAVAWYQQKPGKAPK |
| | | | | LLIYSASFLYSGVPS |
| | | | | RFSGSRSGTDFTLTI |
| | | | | SSLQPEDFATYYCQQ |
| | | | | HYTTPPTFGQGTKVE |
| | | | | IKRTVAAPSVFIFPP |
| | | | | SDEQLKSGTASVVCL |
| | | | | LNNFYPREAKVQWKV |
| | | | | DNALQSGNSQESVTE |
| | | | | QDSKDSTYSLSSTLT |
| | | | | LSKADYEKHKVYACE |
| | | | | VTHQGLSSPVTKSFN |
| | | | | RGEC |
| 18 | Antibody 2265- | Light | | GACATTCAAATGACC |
| | F02 | Chain | | CAGTCTCCGTCGTCA |
| | | | | CTGTCCGCATCCGTT |
| | | | | GGCGACCGCGTTACC |
| | | | | ATCACGTGCCGTGCG |
| | | | | TCGCAAGATGTGAAC |
| | | | | ACCGCCGTGGCGTGG |
| | | | | TATCAGCAAAAACCG |
| | | | | GGCAAAGCTCCGAAG |
| | | | | CTGCTGATCTATTCA |
| | | | | GCCTCTTTCCTGTAC |
| | | | | TCGGGTGTTCCGTCC |
| | | | | CGTTTCTCAGGCTCT |
| | | | | CGCTCGGGTACGGAT |
| | | | | TTCACCCTGACTATT |
| | | | | TCTTCACTGCAACCG |
| | | | | GAAGATTTTGCGACG |
| | | | | TACTACTGTCAGCAG |
| | | | | CATTACACGACTCCG |
| | | | | CCGACCTTTGGTCAG |
| | | | | GGTACCAAGGTCGAG |
| | | | | ATTAAGCGTACCGTG |
| | | | | GCTGCACCATCCGTG |
| | | | | TTTATCTTCCCTCCG |
| | | | | TCTGATGAGCAGCTG |
| | | | | AAATCCGGTACGGCG |
| | | | | TCGGTCGTCTGCTTG |
| | | | | CTGAATAACTTCTAT |
| | | | | CCGCGTGAAGCGAAG |
| | | | | GTGCAATGGAAGGTT |
| | | | | GACAATGCCCTGCAG |
| | | | | TCAGGTAACTCCCAA |
| | | | | GAGTCTGTTACCGAA |
| | | | | CAAGATTCGAAAGAC |
| | | | | TCAACCTACTCCCTG |
| | | | | TCTTCGACGCTGACG |
| | | | | TTGTCCAAAGCGGAC |
| | | | | TATGAGAAACACAAG |
| | | | | GTTTACGCATGTGAA |
| | | | | GTGACCCACCAGGGC |
| | | | | CTGTCATCTCCGGTC |
| | | | | ACCAAATCATTTAAT |
| | | | | CGCGGTGAGTGC |
| 19 | Human IgG1 HC | | | ASTKGPSVFPLAPSS |
| | Constant | | | KSTSGGTAALGCLVK |
| | | | | DYFPEPVTVSWNSGA |
| | | | | LTSGVHTFPAVLQSS |
| | | | | GLYSLSSVVTVPSSS |
| | | | | LGTQTYICNVNHKPS |
| | | | | NTKVDKKVEPKSCDK |
| | | | | THTCPPCPAPELLGG |
| | | | | PSVFLFPPKPKDTLM |
| | | | | ISRTPEVTCVVVDVS |
| | | | | HEDPEVKFNWYVDGV |
| | | | | EVHNAKTKPREEQYN |
| | | | | STYRVVSVLTVLHQD |
| | | | | WLNGKEYKCKVSNKA |
| | | | | LPAPIEKTISKAKGQ |
| | | | | PREPQVYTLPPSREE |
| | | | | MTKNQVSLTCLVKGF |
| | | | | YPSDIAVEWESNGQP |
| | | | | ENNYKTTPPVLDSDG |
| | | | | SFFLYSKLTVDKSRW |
| | | | | QQGNVFSCSVMHEAL |
| | | | | HNHYTQKSLSLSPGK |
| 20 | Human IgG LC | | | RTVAAPSVFIFPPSD |
| | Constant Ckappa | | | EQLKSGTASVVCLLN |
| | | | | NFYPREAKVQWKVDN |
| | | | | ALQSGNSQESVTEQD |
| | | | | SKDSTYSLSSTLTLS |
| | | | | KADYEKHKVYACEVT |
| | | | | HQGLSSPVTKSFNRG |
| | | | | EC |
| 21 | Mouse IgG1 HC | | | AKTTPPSVYPLAPGS |
| | Constant | | | AAQTNSMVTLGCLVK |
| | | | | GYFPEPVTVTWNSGS |
| | | | | LSSGVHTFPAVLQSD |
| | | | | LYTLSSSVTVPSSTW |
| | | | | PSETVTCNVAHPASS |
| | | | | TKVDKKIVPRDCGCK |
| | | | | PCICTVPEVSSVFIF |
| | | | | PPKPKDVLTITLTPK |
| | | | | VTCVVVDISKDDPEV |
| | | | | QFSWFVDDVEVHTAQ |
| | | | | TQPREEQFNSTFRSV |
| | | | | SELPIMHQDWLNGKE |
| | | | | FKCRVNSAAFPAPIE |
| | | | | KTISKTKGRPKAPQV |
| | | | | YTIPPPKEQMAKDKV |
| | | | | SLTCMITDFFPEDIT |
| | | | | VEWQWNGQPAENYKN |
| | | | | TQPIMDTDGSYFVYS |
| | | | | KLNVQKSNWEAGNTF |
| | | | | TCSVLHEGLHNHHTE |
| | | | | KSLSHSPG |
| 22 | Mouse IgG LC | | | RADAAPTVSIFPPSS |
| | Constant | | | EQLTSGGASVVCFLN |
| | Ckappa | | | NFYPKDINVKWKIDG |
| | | | | SERQNGVLNSWTDQD |
| | | | | SKDSTYSMSSTLTLT |
| | | | | KDEYERHNSYTCEAT |
| | | | | HKTSTSPIVKSFNRN |
| | | | | EC |
| 23 | Kappa LC | | | HMTVAAPSVFIFPPS |
| | | | | DEQLKSGTASVVCLL |
| | | | | NNFYPREAKVQWKVD |
| | | | | NALOSGNSQESVTEQ |
| | | | | DSKDSTYSLSSTLTL |
| | | | | SKADYEKHKVYACEV |
| | | | | THQGLSSPVTKSFNR |
| | | | | GEC |
| 24 | Lambda LD | | | GQPKAAPSVTLFPPS |
| | | | | SEELOANKATLVCLI |
| | | | | SDFYPGAVTVAWKAD |
| | | | | SSPVKAGVETTTPSK |
| | | | | QSNNKYAASSYLSLT |
| | | | | PEQWKSHRSYSCQVT |
| | | | | HEGSTVEKTVAPTEC |
| | | | | S |
| 25 | FlagHis Tag | | | GSGDYKDDDDKGSGH |
| | | | | HHHHH |
| 26 | Linker | | | GGGGSGGGGSGGGGS |
| 27 | Linker | | | AAGSDQEPKSS |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

One or more features from any embodiments described herein or in the figures may be combined with one or more features of any other embodiments described herein or in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An antibody conjugate according to the formula: 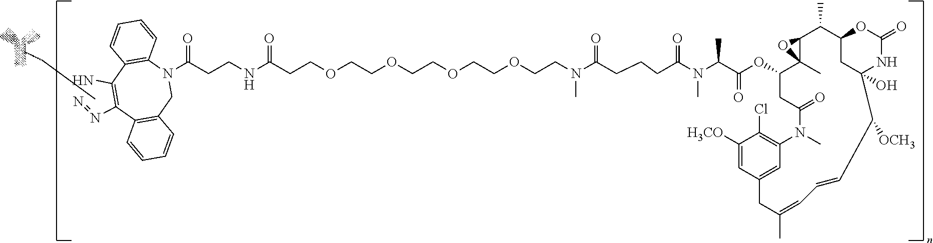
wherein n is from 1 to 4;
the antibody comprises a V_{H} region of SEQ ID NO: 13, and a V_{L} region of SEQ ID NO: 14;
the antibody further comprises a residue of p-azidomethyl-phenylalanine substituting at each of sites HC-F404 and HC-Y180 according to the EU numbering scheme; and
each structure within the brackets of the formula is bonded to the antibody at one of the p-azidomethyl-phenylalanine residues.

2. The antibody conjugate of claim 1 wherein n is 1.

3. The antibody conjugate of claim 1 wherein n is 2.

4. The antibody conjugate of claim 1 wherein n is 3.

5. The antibody conjugate of claim 1 wherein n is 4.

6. The antibody conjugate of any one of the previous claims, further comprising at least one constant region domain.

7. The antibody conjugate of claim 6, wherein the constant region comprises a sequence selected from SEQ ID NO: 19 and 20, or both.

8. The antibody conjugate of any one of the preceding claims, wherein the antibody is a monoclonal antibody.

9. The antibody conjugate of any one of the preceding claims, wherein the antibody is an IgA, an IgD, an IgE, an IgG, or an IgM.

10. The antibody conjugate of any one of the preceding claims, wherein the antibody is humanized or human.

11. The antibody conjugate of any one of the preceding claims, wherein the antibody is aglycosylated.

12. The antibody conjugate of any one of the preceding claims, wherein the antibody is an antibody fragment.

13. The antibody conjugate of claim 12, wherein the antibody fragment is selected from an Fv fragment, a Fab fragment, a F(ab′)₂ fragment, a Fab′ fragment, an scFv (sFv) fragment, and an scFv-Fc fragment.

14. The antibody conjugate of claim 13, wherein the antibody is an scFv fragment.

15. The antibody conjugate of claim 13, wherein the antibody is an scFv-Fc fragment.

16. The antibody conjugate of any one of the preceding claims, wherein the antibody specifically binds cynomolgus BCMA receptor.

17. The antibody conjugate of any one of the preceding claims, wherein the antibody specifically binds mouse BCMA receptor.

18. A kit comprising an antibody conjugate of any one of the preceding claims, and instructions for use of the antibody conjugate.

19. The kit of claim 18, wherein the antibody conjugate is lyophilized.

20. The kit of claim 19, further comprising a fluid for reconstitution of the lyophilized antibody.

21. A pharmaceutical composition comprising the antibody conjugate of any one of claims 1 to 17 and a pharmaceutically acceptable carrier.

22. A method of treating or preventing a disease or condition in a subject in need thereof, comprising administering to the subject an effective amount of the antibody conjugate of any one of claims 1 to 17, or the pharmaceutical composition of claim 21.

23. A method of diagnosing a disease or condition in a subject in need thereof, comprising administering to the subject an effective amount of the antibody conjugate of any one of claims 1 to 17, or the pharmaceutical composition of claim 21.

24. The method of claims 22 to 23, wherein the disease or condition is a cancer.

25. The method of any one of claims 22 to 24, wherein the disease or condition is leukemia.

26. The method of any one of claims 22 to 25, wherein the disease or condition is lymphoma.

27. The method of any one of claims 22 to 24, wherein the disease or condition is multiple myeloma.

28. The method of claim 27, wherein said multiple myeloma is Stage I according to the International Staging System or the Revised International Staging System.

29. The method of claim 27, wherein said multiple myeloma is Stage II according to the International Staging System or the Revised International Staging System.

30. The method of claim 27, wherein said multiple myeloma is Stage III according to the International Staging System or the Revised International Staging System.

31. The method of claim 27, wherein said multiple myeloma is newly-diagnosed multiple myeloma.

32. The method of claim 27, wherein said multiple myeloma is relapsed or refractory multiple myeloma.
